(19) United States
(12) Patent Application Publication
(10) Pub. No.: US 2022/0346456 A1
(43) Pub. Date: Nov. 3, 2022

(54) AEROSOL DELIVERY APPARATUS

(71) Applicant: Nerudia Limited, Livepool (GB)

(72) Inventors: Ian STUART, Liverpool (GB); Jonathan MARCHBANK, Liverpool (GB)

(21) Appl. No.: 17/813,531

(22) Filed: Jul. 19, 2022

Related U.S. Application Data
(63) Continuation of application No. PCT/EP21/52185, filed on Jan. 29, 2021.

(30) Foreign Application Priority Data
Jan. 30, 2020 (EP) 20154504.3
Jan. 30, 2020 (EP) 20154506.8
Jan. 30, 2020 (EP) 20154511.8
Jan. 30, 2020 (EP) 20154513.4
Jan. 30, 2020 (EP) 20154522.5

(51) Int. Cl. A24F 40/485; A24F 40/10; A24F 40/42; A24F 40/46

(52) U.S. Cl. CPC A24F 40/485; A24F 40/10; A24F 40/42; A24F 40/46; A61M 15/002

(57) ABSTRACT

A smoking substitute apparatus comprising an air passage, a reservoir formed from an air-permeable substrate and arranged to allow air to be drawn through the reservoir, the reservoir being loaded with a source of nicotine, and one or more one-way valves arranged along the air passage and configured to allow air to flow along the air passage in an upstream to downstream direction.

### CROSS-REFERENCE TO RELATED APPLICATIONS/INCORPORATION BY REFERENCE STATEMENT

This application is a non-provisional application claiming benefit to the international application no. PCT/EP2021/052185 filed on Jan. 29, 2021, which claims priority to EP 20154522.5 filed on Jan. 30, 2020, to EP 20154504.3 filed on Jan. 30, 2020, to EP 20154506.8 filed on Jan. 30, 2020, to EP 20154511.8 filed on Jan. 30, 2020, and to EP 20154513.4 filed on Jan. 30, 2020. The entire contents of each of the above-referenced applications are hereby incorporated herein by reference in their entirety.

### FIELD OF THE DISCLOSURE

The present disclosure relates to an aerosol delivery apparatus. Such an apparatus is of particular, but not necessary exclusive interest as a smoking substitute apparatus. It is a preferred feature of operation of the apparatus that it is able to deliver an active ingredient (such as nicotine) to a user for inhalation without producing a visible vapour cloud.

### BACKGROUND

The smoking of tobacco is generally considered to expose a smoker to potentially harmful substances. It is thought that a significant amount of the potentially harmful substances are generated through the burning and/or combustion of the tobacco and the constituents of the burnt tobacco in the tobacco smoke itself.

Low temperature combustion of organic material such as tobacco is known to produce tar and other potentially harmful by-products. There have been proposed various smoking substitute systems in which the conventional smoking of tobacco is avoided.

Such smoking substitute systems can form part of nicotine replacement therapies aimed at people who wish to stop smoking and overcome a dependence on nicotine.

Known smoking substitute systems include electronic systems that permit a user to simulate the act of smoking by producing an aerosol (also referred to as a “vapour”) that is drawn into the lungs through the mouth (inhaled) and then exhaled. The inhaled aerosol typically bears nicotine and/or a flavourant without, or with fewer of, the health risks associated with conventional smoking.

In general, smoking substitute systems are intended to provide a substitute for the rituals of smoking, whilst providing the user with a similar, or improved, experience and satisfaction to those experienced with conventional smoking and with combustible tobacco products.

The popularity and use of smoking substitute systems has grown rapidly in the past few years. Such systems may be used as an aid to assist habitual smokers wishing to quit tobacco smoking. There are a number of different categories of smoking substitute systems, each utilising a different smoking substitute approach. Some smoking substitute systems are designed to resemble a conventional cigarette and are cylindrical in form with a mouthpiece at one end. Other smoking substitute devices do not generally resemble a cigarette (for example, the smoking substitute device may have a generally box-like form, in whole or in part).

One approach is the so-called “vaping” approach, in which a vaporisable liquid, or an aerosol former, sometimes typically referred to herein as “e-liquid”, is heated by a heating device (sometimes referred to herein as an electronic cigarette or “e-cigarette” device) to produce an aerosol vapour which is inhaled by a user. The e-liquid typically includes a base liquid, nicotine and may include a flavourant. The resulting vapour therefore also typically contains nicotine and/or a flavourant. The base liquid may include propylene glycol and/or vegetable glycerine.

A typical e-cigarette device includes a mouthpiece, a power source (typically a battery), a tank for containing e-liquid and a heating device. In use, electrical energy is supplied from the power source to the heating device, which heats the e-liquid to produce an aerosol (or “vapour”) which is inhaled by a user through the mouthpiece.

E-cigarettes can be configured in a variety of ways. For example, there are “closed system” vaping smoking substitute systems, which typically have a sealed tank and heating element. The tank is pre-filled with e-liquid and is not intended to be refilled by an end user. One subset of closed system vaping smoking substitute systems include a main body which includes the power source, wherein the main body is configured to be physically and electrically couplable to a consumable including the tank and the heating element. In this way, when the tank of a consumable has been emptied of e-liquid, that consumable is removed from the main body and disposed of. The main body can then be reused by connecting it to a new, replacement, consumable. Another subset of closed system vaping smoking substitute systems are completely disposable, and intended for one-use only.

There are also “open system” vaping smoking substitute systems which typically have a tank that is configured to be refilled by a user. In this way the entire device can be used multiple times.

An example vaping smoking substitute system is the myblu™e-cigarette. The myblu™e-cigarette is a closed system which includes a main body and a consumable. The main body and consumable are physically and electrically coupled together by pushing the consumable into the main body. The main body includes a rechargeable battery. The consumable includes a mouthpiece and a sealed tank which contains e-liquid. The consumable further includes a heater, which for this device is a heating filament coiled around a portion of a wick. The wick is partially immersed in the e-liquid, and conveys e-liquid from the tank to the heating filament. The system is controlled by a microprocessor on board the main body. The system includes a sensor for detecting when a user is inhaling through the mouthpiece, the microprocessor then activating the device in response. When the system is activated, electrical energy is supplied from the power source to the heating device, which heats e-liquid from the tank to produce a vapour which is inhaled by a user through the mouthpiece.

An alternative to the “vaping” approach is the so-called Heated Tobacco (“HT”) approach in which tobacco (rather than an e-liquid) is heated or warmed to release vapour. HT is also known as “heat not burn” (“HNB”). The tobacco may be leaf tobacco or reconstituted tobacco. In the HT approach the intention is that the tobacco is heated but not burned, i.e., the tobacco does not undergo combustion.

The heating, as opposed to burning, of the tobacco material is believed to cause fewer, or smaller quantities, of the more harmful compounds ordinarily produced during conventional smoking. Consequently, the HT approach may reduce the odour and/or health risks that can arise through the burning, combustion and pyrolytic degradation of tobacco.

A typical HT smoking substitute system may include a device and a consumable. The consumable may include the tobacco material. The device and consumable may be configured to be physically coupled together. In use, heat may be imparted to the tobacco material by a heating element of the device, wherein airflow through the tobacco material causes components in the tobacco material to be released as vapour. A vapour may also be formed from a carrier in the tobacco material (this carrier may for example include propylene glycol and/or vegetable glycerine) and additionally volatile compounds released from the tobacco. The released vapour may be entrained in the airflow drawn through the tobacco.

As the vapour passes through the consumable (entrained in the airflow) from the location of vapourisation to an outlet of the consumable (e.g., a mouthpiece), the vapour cools and condenses to form an aerosol for inhalation by the user. The aerosol may contain nicotine and/or flavour compounds.

A further alternative to vaping-type smoking substitute systems and HNB systems is an inhaler apparatus, of which a particular example is the Nicorette® inhalator (trade name). Such systems are often passive in the sense that they do not require a source of heat or other activation energy in order to generate a vapour. As with an e-cigarette, such an inhaler typically includes a mouthpiece and a main body containing a source of nicotine. In use, a user may inhale or “puff” on the mouthpiece to draw air over or through the nicotine source. The nicotine source may be, for example, an air-permeable substrate impregnated with nicotine. When the supply of nicotine in the nicotine source is depleted, such that the user no longer receives sufficient (or any) nicotine with each puff, the user can replace the nicotine source in order to continue nicotine delivery.

### SUMMARY OF THE DISCLOSURE

Smoking substitute systems (e.g., e-cigarettes) are generally regarded as having fewer of the health risks associated with conventional smoking, not only for the user themselves, but also for those nearby (i.e., those affected by passive smoking). However, the exhaling of a visible vapour cloud by the user may still be regarded in some circumstances as socially unacceptable, in light of the negative views surrounding smoking itself. Therefore, many locations where smoking is not permitted also do not allow smoking substitute systems, instead requiring users to use the same designated smoking areas as smokers themselves. This can reduce the attractiveness of smoking substitute systems, and reduce their uptake as a smoking cessation aid.

Accordingly, it would be advantageous to provide a smoking substitute system that can provide a similar user experience to an e-cigarette, but without producing a visible vapour cloud. Alternatively, or additionally, it would be advantageous to provide a smoking substitute system that can allow a user to select whether or not the system produces a vapour cloud by switching between one system operating mode where a vapour cloud is produced and another system operating mode where a vapour cloud is not produced.

Still further, based on the insight of the present inventors, it would be advantageous to provide an aerosol delivery system, not necessarily limited to a smoking substitute system, for the delivery of an active ingredient to a user by inhalation, providing the beneficial effects referred to above.

The present disclosure (Development A) has been devised in the light of the above considerations.

Development A: An Aerosol Delivery Apparatus Comprising an Air Permeable Reservoir and at Least One-Way Valve Arranged in the Air Passage

In a general aspect of Development A, the present disclosure relates to an aerosol delivery apparatus comprising an air permeable reservoir and at least one-way valve arranged in the air passage, the one or more one-way valves being provided to control air-flow through the air passage.

According to a first preferred aspect of Development A there is provided an aerosol delivery apparatus comprising an air passage, a reservoir formed from an air-permeable substrate and arranged to allow air to be drawn through the reservoir, the reservoir being loaded with a source of an active ingredient, and one or more one-way valves arranged along the air passage and configured to allow air to flow along the air passage in an upstream to downstream direction.

Providing at least one one-way valve in the air passage can reduce, substantially prevent or prevent air flow along the air passage in a downstream to upstream direction, controlling flow of both air and vapour through the passage and apparatus. Furthermore, the incorporation of a one-way valve may allow there to be a threshold pressure difference below which air flow will be blocked by the valve even in the upstream to downstream direction. This can ensure that the one-way valve is only opened for example when a user draws air through the apparatus. Such an arrangement can serve to prolong the useful lifetime of the reservoir.

In some embodiments, the aerosol delivery apparatus is a smoking substitute apparatus. In such embodiments, the active ingredient may comprise or consist of nicotine.

Optionally, at least one of the one or more one-way valves may be arranged upstream of the reservoir along the air passage.

Conveniently, at least one of the one or more one-way valves may be arranged downstream of the reservoir along the air passage.

Advantageously, the apparatus may comprise a first one-way valve and a second one-way valve arranged within the air passage, wherein the first one-way valve is arranged upstream of the reservoir along the air passage, and the second one-way valve is arranged downstream of the reservoir along the air passage.

Optionally, the aerosol delivery apparatus may further comprise a heater arranged in the air passage and upstream of the reservoir, the heater being operable to heat air passing through the air passage.

Conveniently, the heater may comprise an electrically heatable mesh.

Advantageously, the heater may comprise an electrically heatable heating coil.

Optionally, the heater may be heatable by resistive heating using an electrical current.

Conveniently, a one-way valve of the one-way valves may be arranged upstream of the heater along the air passage.

Advantageously, a one-way valve of the one-way valves may be a duckbill valve.

Optionally, a one-way valve of the one-way valves may be a ball one-way valve.

Conveniently, the ball one-way valve may further comprise a spring to locate the ball within the valve.

Advantageously, the combined resistance to draw presented by the one-way valve, reservoir and air passage may be substantially equal to that of a conventional cigarette.

Optionally, the reservoir is a consumable component of the aerosol delivery apparatus.

According to a second preferred aspect of Development A, the aerosol delivery apparatus is comprised by or within a cartridge configured for engagement with a base unit, the cartridge and base unit together forming an aerosol delivery system.

According to a third preferred aspect of Development A, there is provided an aerosol delivery system comprising a base unit, and an aerosol delivery apparatus according to the second aspect of Development A, wherein the aerosol delivery apparatus is removably engageable with the base unit.

According to a fourth preferred aspect of Development A, there is provided a method of using an aerosol delivery apparatus according to the first or second aspects of Development A to generate an aerosol.

There now follows a disclosure of various optional features. These are intended to be applicable to Development A, disclosed above, and may also be applied in any combination (unless the context demands otherwise) to any aspect, embodiment or optional feature set out with respect to Development B, Development C, Development D and/or Development E.

The aerosol delivery apparatus may be in the form of a consumable. The consumable may be configured for engagement with a main body. When the consumable is engaged with the main body, the combination of the consumable and the main body may form an aerosol delivery system such as a closed aerosol delivery system. For example, the consumable may comprise components of the system that are disposable, and the main body may comprise non-disposable or non-consumable components (e.g., power supply, controller, sensor, etc.) that facilitate the generation and/or delivery of aerosol by the consumable. In such an embodiment, an aerosol precursor (e.g., e-liquid) and/or other nicotine source (e.g., nicotine-infused air-permeable substrate) may be replenished by replacing a used consumable with an unused consumable.

Alternatively, the aerosol delivery apparatus may be a non-consumable apparatus (e.g., that is in the form of an open aerosol delivery system). In such embodiments an aerosol former (e.g., e-liquid) of the system may be replenished by re-filling, e.g., a reservoir of the aerosol delivery apparatus, with the aerosol precursor (rather than replacing a consumable component of the apparatus). In some embodiments, it may only be a nicotine source itself that is replaced. The aerosol delivery apparatus may therefore comprise a space or receptacle into which a nicotine source (e.g., nicotine-infused air-permeable substrate) may be placed.

In light of this, it should be appreciated that some of the features described herein as being part of the aerosol delivery apparatus may alternatively form part of a main body for engagement with the aerosol delivery apparatus. This may be the case in particular when the aerosol delivery apparatus is in the form of a consumable.

Where the aerosol delivery apparatus is in the form of a consumable, the main body and the consumable may be configured to be physically coupled together. For example, the consumable may be at least partially received in a recess of the main body, such that there is an interference fit between the main body and the consumable. Alternatively, the main body and the consumable may be physically coupled together by screwing one onto the other, or through a bayonet fitting, or the like.

Thus, the aerosol delivery apparatus may comprise one or more engagement portions for engaging with a main body. In this way, one end of the aerosol delivery apparatus may be coupled with the main body, whilst an opposing end of the aerosol delivery apparatus may define a mouthpiece of the aerosol delivery system.

In order to generate an aerosol, the reservoir (or air-permeable substrate, as appropriate) comprises at least one volatile compound that is intended to be vaporised/aerosolised and that may provide the user with a recreational, wellness, nutritional, physiological and/or medicinal effect when inhaled. Such a volatile compound is referred to herein as an “active agent” or “active ingredient”.

The active ingredient may comprise or consist of nicotine. However, in some embodiments, the active ingredient may not comprise nicotine, and may instead comprise or consist of one or more of a nutritional agent, a pharmaceutical agent or a flavour agent.

Suitable active agents include the group consisting of: nicotine, cocaine, caffeine (anhydrous or salts thereof), vitamins, minerals, amino acids, plant or herbal concentrated extracts, sugars, opiates and opioids, cathine and cathinone, kavalactones, mysticin, beta-carboline alkaloids, salvinorin A, cannabinoids, phytocannabinoids, one or more flavourants, together with any combinations, functional equivalents to, and/or synthetic alternatives of the foregoing.

Example flavourants may include menthol, liquorice, chocolate, fruit flavour (including, e.g., citrus, cherry etc.), vanilla, spice (e.g., ginger, cinnamon) and tobacco flavour.

Cannabinoid compounds include phyto-cannabinoids which include: cannabidiol (CBD) and its derivatives/homologues (e.g., cannabidiol mono(m)ethyl ether, cannabidivarin (CBDV), cannabidiorcol, cannabidiolic acid, cannabidivarinic acid); cannabinodiol (CBND) and its derivatives/homologues (e.g., carrabinodivarin); cannabigerol (CBG) and its derivatives/homologues (e.g., cannabigerol mono(m)ethyl ether, cannabinerolic acid A, cannabigerovarin, cannabigerolic acid A, cannabigerolic acid A mono(m)ethyl ether, cannabigerovarinic acid A); cannabinol (CBN) and its derivatives/homologues (e.g., cannabivarin/cannabivarol (CBV), cannabiorcol, cannabinolic acid, cannabinol (m)ethyl ester); tetrahydrocannabinol (THC) and its derivatives/homologues (e.g., tetrahydrocannabivarin (THCV), tetrahydrocannabiorcol, tetrahydrocannabinolic acid A/B, tetrahydrocannabivarinic acid A, tetrahydrocannabiorcolic acid A/B, isotetrahydrocannabinol, isotetrahydrocannabivarin); cannabicyclol (CBL) and its derivatives/homologues (e.g., cannabicyclolic acid, cannabicyclovarin); cannabichromene (CBC) and its derivatives/homologues (e.g., cannabichromenic acid A, cannabichromevarin (CBCV), cannabichromevarinic acid A); cannabielsoin (CBE) and its derivatives/homologues (e.g., cannabielsoic acid A/B, cannabiglendol, dehydrocannabifuran, cannabifuran); cannabicitran (CBT) and its derivatives/homologues; cannabitriol and its derivatives/homologues (e.g., ethyl cannabitriol, dihydroxy-tetrahydrocannabinol, cannabidiolic acid A cannabitriol ester, dihydroxy-hexahydrocannabinol (cannabiripsol), cannabitetrol, oxo-tetrahydrocannabinol); and cannabichromanone (CBCN) and its derivatives/homologues (e.g., cannabicoumaronone).

In some embodiments, the cannabinoid compound is selected from at least one of cannabidiol (CBD) and its derivatives/homologues, e.g., cannabiodiol-C₅ (CBD-C₅), cannabidiol-C₄ (CBD-C₄), cannabidiol mono(m)ethyl ether (CBDM-C₅), cannabidivarin (CBDV-C₃), cannabidiorcol (CBD-C₁), cannabidiolic acid (CBDA-C₅), cannabidivarinic acid (CBDVA-C₃).

In some embodiments, the cannabinoid compound is selected from at least one of tetrahydrocannabinol (THC) and its derivatives/homologues, e.g., Δ⁹-tetrahydrocannabinol (Δ⁹-THC-C₅/cis-Δ⁹-THC-C₅), Δ⁸-tetrahydrocannabinol (Δ⁸-THC-C₅), Δ⁸-tetrahydrocannabinolic acid A (Δ⁸-THCA-C₅ A), Δ⁹-tetrahydrocannabinol-C₄ (Δ⁹-THC-C₄), Δ⁹-tetrahydrocannabivarin (Δ⁹-THCV-C₃), Δ⁹-tetrahydrocannabiorcol (Δ⁹-THCO-C₁), Δ⁹-tetrahydrocannabinolic acid A (Δ⁹-THCA-C₅ A), Δ⁹-tetrahydrocannabinolic acid B (Δ⁹-THCA-C₅ B), Δ⁹-tetrahydrocannabinolic acid-C₄ A and/or B (Δ⁹-THCA-C₄ A and/or B), Δ⁹-tetrahydrocannabivarinic acid A (Δ⁹-THCVA-C₃ A), A⁹-tetrahydrocannabiorcolic acid A and/or B (Δ⁹-THCOA-C₁ A and/or B), isotetrahydrocannabinol and isotetrahydrocannabivarin.

The total amount of cannabinoid compounds in the substrate may be at least 200 mg; for example, it may be at least 250 mg, at least 300 mg, at least 400 mg, at least 500 mg. In some cases, lower amounts may be preferred. The total amount of cannabinoid compounds in the substrate may therefore be at least 10 mg, at least 20 mg, at least 30 mg, at least 40 mg, at least 50 mg, at least 75 mg, at least 100 mg.

In some cases, it may be desirable to limited the total amount of cannabinoid compounds, which may be not more than 200 mg, not more than 175 mg, not more than 150 mg, not more than 125 mg, not more than 100 mg, not more than 75 mg, not more than 50 mg, not more than 40 mg, not more than 30 mg, not more than 20 mg, not more than 10 mg. In some cases, the total amount of the cannabinoid compounds may be not more than 5 mg.

Where THC is included, either as one cannabinoid compound in a mixture or as the only cannabinoid, the total of amount of THC may be limited. In some cases, the total amount of THC in the substrate is not more than 100 mg, not more than 75 mg, not more than 50 mg, not more than 40 mg, not more than 30 mg, not more than 20 mg, not more than 15 mg, not more than 10 mg, not more than 5 mg, not more than 3 mg. In some cases, the amount of THC may be 0.1 to 30 mg, for example 1 to 30 mg, for example 1 to 20 mg, for example 1 to 10 mg, for example 1 to 5 mg, for example 1 to 3 mg.

As already disclosed above, the aerosol delivery apparatus comprises (or may comprise) a reservoir configured to store an aerosol precursor. The aerosol precursor may be formulated so as to produce a non-visible or substantially non-visible vapour. The aerosol precursor may comprise a base liquid. The aerosol precursor may additionally comprise nicotine. The aerosol precursor may be an e-liquid. The aerosol precursor may consist substantially of nicotine or a nicotine compound. The aerosol precursor may further comprise a flavourant. Alternatively, the aerosol precursor may be substantially flavourless. That is, the aerosol precursor may not contain any deliberately added additional flavourant. A flavourant may be provided as a separate flavourant aerosol precursor, such that the aerosol precursor and flavourant aerosol precursor may be separately vaporised to form an aerosol comprising both the aerosol precursor and the flavourant aerosol precursor.

The air-permeable substrate, storing the aerosol precursor and/or the flavourant, may constitute the entirety of the reservoir. The air-permeable substrate may be impregnated with the aerosol precursor and/or the flavourant aerosol precursor. The substrate material may be a foamed polymer which will allow for airflow to pass through the substrate at a given pressure drop value, so as to provide a comfortable ‘draw’ sensation for the user. The substrate may be, for example, a sintered polyethylene or a PET foam.

The substrate may be impregnated with nicotine via immersion in a liquid containing nicotine and a volatile carrier (for example a solution of nicotine in ethanol). The substrate may be immersed to evenly soak the substrate. Once removed and left to dry or baked in an oven, the carrier is evaporated and the nicotine is left evenly spread throughout the substrate.

The aerosol precursor and/or the flavourant aerosol precursor may be formulated to form a vapour when ambient air is drawn through the reservoir. Alternatively, the aerosol precursor and/or the flavourant aerosol precursor may be formulated to form a vapour when heated air is drawn through the reservoir. The reservoir may comprise a monolithic substrate. The reservoir may consist of a plurality of substrates, each arranged to allow air to be drawn therethrough, and each comprising one or both of an aerosol precursor and the flavourant aerosol precursor. The aerosol precursor and/or the flavourant aerosol precursor may be provided in spatially coterminous or spatially distinct regions of the reservoir.

For the avoidance of doubt, it is intended in this disclosure for some embodiments that the “source of nicotine” in the first reservoir region may be different to the “source of flavourant” in the second reservoir region.

The aerosol delivery apparatus may comprise more than one passage for fluid (e.g., air) flow therethrough. Where more than one passage is present, one or more of the passages may be distinct, such that there is no intersection between the passages. One or more of the passages may comprise junctions or openings therebetween such that fluid within the passages can mix within the aerosol delivery apparatus.

The passage through the reservoir comprises one or more valves to control fluid flow. (In some embodiments, one or more of the passages may comprise one or more valves to control fluid flow.) The valves include, or may include, one or more one-way valve to ensure fluid (i.e., air) can only flow through the passage in a desired direction. Further valves may be provided that may be operable to open and close the passage such that fluid is enabled to or prevented from flowing through the passage. More than one such valve may be linked such that the valves may be operated in combination or in synchronism with each other. A valve to open and close a passage may be controlled by mechanical means (i.e., the user moves the valve using a control lever or similar) or by electrical control (i.e., moved in response to a control signal from a processor or control system of the aerosol delivery apparatus).

The passages may extend through (at least a portion of) the aerosol delivery apparatus, between openings that may define an inlet and an outlet of a passage. Each inlet and outlet may be in fluid communication with only one passage, or a subset of the passages, or all the passages in the aerosol delivery apparatus. The outlet or outlets may be at a mouthpiece of the aerosol delivery apparatus. In this respect, a user may draw fluid (e.g., air) into and through a passage by inhaling at the outlet (i.e., using the mouthpiece).

In some embodiments, a passage through the aerosol delivery apparatus may be at least partially defined by a tank forming the aerosol precursor reservoir or part of the aerosol precursor reservoir. The tank may substantially (or fully) define the passage, for at least a part of the length of the passage. In this respect, the tank may surround the passage, e.g., in an annular arrangement around the passage.

One or more of the fluid passages may comprise a heater for heating the fluid (i.e., air) passing through the passage. The heater may, for example, be arranged upstream of a reservoir formed from an air permeable substrate such that air warmed by the heater is drawn through the reservoir to enable or increase nicotine or flavourant vapourisation and subsequent entrainment in the air-flow. The heater to heat the air may comprise one or more meshes arranged within the fluid passage. The heater to heat the air may comprise one or more thermally conductive elements to conduct heat from a heater to the air passage or to increase the heat transfer between the heater and the air. Alternatively, the heat source may be non-electrical. For example, the heat to heat the air may be generated by an exothermic reaction. An exothermic reaction heat source may comprise a single-use (i.e., consumable) reaction container. Alternatively, an exothermic reaction heat source may be rechargeable (e.g., by using a reversible reaction such as a crystallisation process).

The aerosol delivery apparatus (or main body engaged with the aerosol delivery apparatus) may comprise a power source, in some embodiments. The power source may be electrically connected (or connectable) to a heater of the aerosol delivery apparatus (e.g., when the aerosol delivery apparatus is engaged with the main body). The power source may be a battery (e.g., a rechargeable battery). A connector in the form of, e.g., a USB port may be provided for recharging this battery.

When the aerosol delivery apparatus is in the form of a consumable, the aerosol delivery apparatus may comprise an electrical interface for interfacing with a corresponding electrical interface of the main body. One or both of the electrical interfaces may include one or more electrical contacts. Thus, when the main body is engaged with the consumable, the electrical interface of the main body may be configured to transfer electrical power from the power source to a heater of the consumable via the electrical interface of the consumable.

The electrical interface of the aerosol delivery apparatus may also be used to identify the aerosol delivery apparatus (in the form of a consumable) from a list of known types. For example, the consumable may have a certain concentration of nicotine and the electrical interface may be used to identify this. The electrical interface may additionally or alternatively be used to identify when a consumable is connected to the main body.

Again, where the aerosol delivery apparatus is in the form of a consumable, the main body may comprise an identification means, which may, for example, be in the form of an RFID reader, a barcode or QR code reader. This identification means may be able to identify a characteristic (e.g., a type) of a consumable engaged with the main body. In this respect, the consumable may include any one or more of an RFID chip, a barcode or QR code, or memory within which is an identifier and which can be interrogated via the identification means.

The aerosol delivery apparatus or main body may comprise a controller, which may include a microprocessor. The controller may be configured to control the supply of power from the power source to the heater(s) of the aerosol delivery apparatus (e.g., via the electrical contacts). A memory may be provided and may be operatively connected to the controller. The memory may include non-volatile memory. The memory may include instructions which, when implemented, cause the controller to perform certain tasks or steps of a method.

The main body or aerosol delivery apparatus may comprise a wireless interface, which may be configured to communicate wirelessly with another device, for example a mobile device, e.g., via Bluetooth®. To this end, the wireless interface could include a Bluetooth® antenna. Other wireless communication interfaces, e.g., WIFI®, are also possible. The wireless interface may also be configured to communicate wirelessly with a remote server.

A puff sensor may be provided that is configured to detect a puff (i.e., inhalation from a user). The puff sensor may be operatively connected to the controller so as to be able to provide a signal to the controller that is indicative of a puff state (i.e., puffing or not puffing). The puff sensor may, for example, be in the form of a pressure sensor or an acoustic sensor. That is, the controller may control power supply to the heater(s) of the consumable and/or aerosol delivery apparatus in response to a puff detection by the sensor. The control may be in the form of activation of the heater(s) in response to a detected puff. That is, the aerosol delivery apparatus may be configured to be activated when a puff is detected by the puff sensor. When the aerosol delivery apparatus is in the form of a consumable, the puff sensor may be provided in the consumable or alternatively may be provided in the main body. Where multiple independent passages are provided within the aerosol delivery apparatus, each of the passages may have a puff sensor.

The term “flavourant” is used to describe a compound or combination of compounds that provide flavour and/or aroma. For example, the flavourant may be configured to interact with a sensory receptor of a user (such as an olfactory or taste receptor). The flavourant may include one or more volatile substances.

The flavourant may be provided in solid or liquid form. The flavourant may be natural or synthetic. For example, the flavourant may include menthol, liquorice, chocolate, fruit flavour (including, e.g., citrus, cherry etc.), vanilla, spice (e.g., ginger, cinnamon) and tobacco flavour. The flavourant may be evenly dispersed or may be provided in isolated locations and/or varying concentrations.

The first aerosol generator comprises an air-permeable substrate. Or, in some embodiments, there may be only a single aerosol generator. This may comprise an air-permeable substrate. The air-permeable substrate may comprise plant material. The plant material may comprise least one plant material selected from the list including Amaranthus dubius, Arctostaphylos uva-ursi (Bearberry), Argemone mexicana, Amica, Artemisia vulgaris, Yellow Tees, Galea zacatechichi, Canavalia maritima (Baybean), Cecropia mexicana (Guamura), Cestrum noctumum, Cynoglossum virginianum (wild comfrey), Cytisus scoparius, Damiana, Entada rheedii, Eschscholzia califomica (California Poppy), Fittonia albivenis, Hippobroma longiflora, Humulus japonica (Japanese Hops), Humulus lupulus (Hops), Lactuca virosa (Lettuce Opium), Laggera alata, Leonotis leonurus, Leonurus cardiaca (Motherwort), Leonurus sibiricus (Honeyweed), Lobelia cardinalis, Lobelia inflata (Indian-tobacco), Lobelia siphilitica, Nepeta cataria (Catnip), Nicotiana species (Tobacco), Nymphaea alba (White Lily), Nymphaea caerulea (Blue Lily), Opium poppy, Passiflora incamata (Passionflower), Pedicularis densiflora (Indian Warrior), Pedicularis groenlandica (Elephant's Head), Salvia divinorum, Salvia dorrii (Tobacco Sage), Salvia species (Sage), Scutellaria galericulata, Scutellaria lateriflora, Scutellaria nana, Scutellaria species (Skullcap), Sida acuta (Wireweed), Sida rhombifolia, Silene capensis, Syzygium aromaticum (Clove), Tagetes lucida (Mexican Tarragon), Tarchonanthus camphoratus, Tumera diffusa (Damiana), Verbascum (Mullein), Zamia latifolia (Maconha Brava) together with any combinations, functional equivalents to, and/or synthetic alternatives of the foregoing.

In some embodiments, the plant material is tobacco. Any type of tobacco may be used. This includes, but is not limited to, flue-cured tobacco, burley tobacco, Maryland Tobacco, dark-air cured tobacco, oriental tobacco, dark-fired tobacco, perique tobacco and rustica tobacco. This also includes blends of the above-mentioned tobaccos.

Any suitable parts of the tobacco plant may be used. This includes leaves, stems, roots, bark, seeds and flowers.

The tobacco may comprise one or more of leaf tobacco, stem tobacco, tobacco powder, tobacco dust, tobacco derivatives, expanded tobacco, homogenised tobacco, shredded tobacco, extruded tobacco, cut rag tobacco and/or reconstituted tobacco (e.g., slurry recon or paper recon).

The air-permeable substrate may comprise a gathered sheet of homogenised (e.g., paper/slurry recon) tobacco or gathered shreds/strips formed from such a sheet.

In some embodiments, the sheet used to form the aerosol-forming substrate has a grammage greater than or equal to 100 g/m², e.g., greater than or equal to 110 g/m² such as greater than or equal to 120 g/m².

The sheet may have a grammage of less than or equal to 300 g/m², e.g., less than or equal to 250 g/m² or less than or equal to 200 g/m².

The sheet may have a grammage of between 120 and 190 g/m².

The air-permeable substrate may comprise at least 50 wt % plant material, e.g., at least 60 wt % plant material, e.g., around 65 wt % plant material. The air-permeable substrate may comprise 80 wt % or less plant material, e.g., 75 or 70 wt % or less plant material.

The air-permeable substrate may comprise one or more additives selected from flavourants, fillers and binders.

Typically, the air-permeable substrate does not comprise a humectant. Humectants may be provided in heat not burn (HNB) tobacco charges. In such cases, humectants are provided as vapour generators, the generated vapour being used to help carry volatile active compounds and to increase visible vapour. Accordingly, it is preferred that the air-permeable substrate does not comprise one or more humectants such as polyhydric alcohols (e.g., propylene glycol (PG), triethylene glycol, 1,2-butane diol and vegetable glycerine (VG)) and their esters (e.g., glycerol mono-, di- or tri-acetate). If such humectants are present in the air-permeable substrate, they may be present at a low level, such as less than 0.5 wt %, more preferably less than 0.1 wt.

Suitable binders are known in the art and may act to bind together the components forming the air-permeable substrate. Binders may comprise starches and/or cellulosic binders such as methyl cellulose, ethyl cellulose, hydroxypropyl cellulose, hydroxyethyl cellulose and methyl cellulose, gums such as xanthan, guar, Arabic and/or locust bean gum, organic acids and their salts such as alginic acid/sodium alginate, agar and pectins.

Preferably the binder content is 5 to 10 wt % of the air-permeable substrate, e.g., around 6 to 8 wt %.

Suitable fillers are known in the art and may act to strengthen the air-permeable substrate. Fillers may comprise fibrous (non-tobacco) fillers such as cellulose fibres, lignocellulose fibres (e.g., wood fibres), jute fibres and combinations thereof.

Preferably, the filler content is 5 to 10 wt % of the aerosol-forming substrate, e.g., around 6 to 9 wt %.

The air-permeable substrate may comprise an aqueous and/or non-aqueous solvent. In some embodiments, the air-permeable substrate has a water content of between 4 and 10 wt %, e.g., between 6-9 wt % such as between 7-9 wt %. Such low moisture content in the air-permeable substrate typically has the effect that, when the air-permeable substrate is exposed to heated air, there would typically not be produced a substantial visible vapour. It is to be noted that in one embodiment it is possible to use as the air-permeable substrate a low moisture tobacco material with its natural nicotine content. The natural nicotine content then meets the requirements of the active agent.

The air-permeable substrate may be at least partly circumscribed by a wrapping layer, e.g., a paper wrapping layer. The wrapping layer may overlie an inner foil layer or may comprise a paper/foil laminate (with the foil innermost).

The plant material may comprise cannabis plant material including Cannabis sativa, Cannabis indica and Cannabis rudealis. The plant material may comprise Echinacea purpurea, Echinacea angustifolia, Acmella oleracea, Helichrysum umbraculigerum, or Radula marginata. This also includes blends of the above-mentioned plant material.

In some embodiments, the cannabinoid-containing plant material is cannabis. The plant may be a traditional strain, or may be a strain bred or other modified (e.g., genetically) to produce certain levels of some cannabinoids compounds, e.g., low levels of THC or high levels of THC.

Any suitable parts of the cannabinoid-containing plant may be used. Thus the cannabinoid-containing plant material may comprise leaves, stems, roots, bark, seeds, buds and flowers (which may be cured).

Development B: An Aerosol Delivery System with a Non-Electrical Heat Source

Smoking substitute systems (e.g., e-cigarettes) are generally regarded as having fewer of the health risks associated with conventional smoking, not only for the user themselves, but also for those nearby (i.e., those affected by passive smoking). However, the exhaling of a visible vapour cloud by the user may still be regarded in some circumstances as socially unacceptable, in light of the negative views surrounding smoking itself. Therefore, many locations where smoking is not permitted also do not allow smoking substitute systems, instead requiring users to use the same designated smoking areas as smokers themselves. This can reduce the attractiveness of smoking substitute systems, and reduce their uptake as a smoking cessation aid.

Accordingly, it would be advantageous to provide a smoking substitute system that can provide a similar user experience to an e-cigarette, but without producing a visible vapour cloud. Alternatively, or additionally, it would be advantageous to provide a smoking substitute system that can allow a user to select whether or not the system produces a vapour cloud by switching between one system operating mode where a vapour cloud is produced and another system operating mode where a vapour cloud is not produced.

Still further, based on the insight of the present inventors, it would be advantageous to provide an aerosol delivery system, not necessarily limited to a smoking substitute system, for the delivery of an active ingredient to a user by inhalation, providing the beneficial effects referred to above.

The present disclosure (Development B) has been devised in the light of the above considerations.

In a general aspect of Development B, the present disclosure relates to an aerosol delivery system with a non-electrical heat source, wherein an air passage around or through the heat source follows an extended path.

According to a first preferred aspect of Development B there is provided an aerosol delivery system comprising an air inlet, an outlet, and an air passage extending from the air inlet to the outlet. A part of the air passage is defined by a heat source air passage extending through a heat source operable to heat air in the heat source air passage through an exothermic reaction. A reservoir formed from an air-permeable substrate is arranged to allow air in the air passage drawn from the outlet of the air passage to be drawn through the reservoir and on to the outlet, the reservoir being loaded with a source of an active ingredient. The heat source air passage has a heat source air inlet and a heat source air outlet, spaced apart on the heat source by a separation distance, wherein, in use, the heat source air passage conveys air along a flow path within the heat source from the heat source air inlet to the heat source air outlet, and wherein the length of the flow path is greater than the separation distance.

In some embodiments, the aerosol delivery system is a smoking substitute system. In such embodiments, the active ingredient may comprise or consist of nicotine.

The provision of a heat source air passage of this configuration allows improved heating of the air flowing through the heat source air passage, by providing a greater flow path length proximate to the heat source.

Optionally, the length of the flow path may be not less than 1.1 times the separation distance, preferably not less than 1.5 times the separation distance, not less than 2 times the separation distance, not less than 5 times the separation distance or not less than 10 times the separation distance.

A higher ratio of flow path length to separation distance can improve the heat transfer between the heat source and the air in the heat source passage.

Conveniently, the length of the flow path may be less than 100 times the separation distance, preferably less than 80 times the separation distance, or less than 50 times the separation distance, or less than 20 times the separation distance.

A lower ratio of flow path length to separation distance can provide a less restrictive flow path, by allowing a larger cross-sectional area configuration for the heat source air passage, reducing the effect of the heat source air passage on the overall resistance to draw for the smoking substitute apparatus.

Advantageously, the heat source air passage conveys air along a tortuous flow path. A torturous flow path may comprise one or more loops or turns or corners or may be, for example, a helical flow path, or a serpentine flow path.

Optionally, the aerosol delivery system may comprises a plurality of separate heat source air passages. At least two of the heat source air passages may extend from a common heat source air inlet. At least two of the heat source air passages may extend to a common heat source air outlet.

Optionally, the exothermic reaction of the heat source may be a crystallisation of a super-saturated solution. The exothermic reaction of the heat source may be initiable by operation of a multistable trigger element or bistable trigger element. Advantageously, the bistable trigger element might be a metallic disc which is operable by inversion from a convex configuration to a concave configuration and/or a concave configuration to a convex configuration. The metallic disc may be invertable using a lever movable in contact with the metallic disc.

In some embodiments, the exothermic reaction of the heat source may be an oxidation reaction. The oxidation reaction may be a reaction between iron powder and oxygen. The oxygen may be atmospheric oxygen. Conveniently, the exothermic reaction may be initiable by removal of an oxygen impermeable seal from a reaction chamber of the heat source.

In some embodiments, the exothermic reaction of the heat source may be a reaction between two or more substances stored inside the heat source and initiable by contact between the two or more substances. The contact between the two or more substances may comprise mixing of the two substances. Such mixing may be by diffusion. Additionally, or alternatively, the mixing may be caused or improved by agitation of the heat source, for example by shaking of the heat source or smoking substitute system.

In some embodiments, the heat source may be a replaceable component of the smoking substitute system.

According to a second preferred aspect of Development B of the disclosure, there is provided a kit of parts for an aerosol delivery system, comprising a base unit, an aerosol delivery apparatus removably engageable with the base unit, and the heat source. The aerosol delivery apparatus is a cartridge configured for engagement with the base unit, the cartridge comprising the reservoir.

Optionally, in use, the heat source may be comprised within the base unit.

Conveniently, in use, the heat source may be comprised within the aerosol delivery apparatus.

According to a third preferred aspect of Development B of the disclosure, there is provided a method of using an aerosol delivery system according to the first aspect of Development B, comprising the steps of initiating the exothermic reaction, and drawing air through the passage to generate heated air in the heat source air passage and through the reservoir to generate a vapour.

The aerosol delivery apparatus may be in the form of a consumable. The consumable may be configured for engagement with a main body. When the consumable is engaged with the main body, the combination of the consumable and the main body may form an aerosol delivery system such as a closed smoking substitute system. For example, the consumable may comprise components of the system that are disposable, and the main body may comprise non-disposable or non-consumable components (e.g., power supply, controller, sensor, etc.) that facilitate the generation and/or delivery of aerosol by the consumable. In such an embodiment, an aerosol precursor (e.g., e-liquid) and/or other nicotine source (e.g., nicotine-infused air-permeable substrate) may be replenished by replacing a used consumable with an unused consumable.

Alternatively, the aerosol delivery apparatus may be a non-consumable apparatus (e.g., that is in the form of an open aerosol delivery system). In such embodiments an aerosol former (e.g., e-liquid) of the system may be replenished by re-filling, e.g., a reservoir of the aerosol delivery apparatus, with the aerosol precursor (rather than replacing a consumable component of the apparatus).

In light of this, it should be appreciated that some of the features described herein as being part of the aerosol delivery apparatus may alternatively form part of a main body for engagement with the aerosol delivery apparatus. This may be the case in particular when the aerosol delivery apparatus is in the form of a consumable.

Development C: An Aerosol Delivery Apparatus Comprising a First Reservoir Region Formed from an Air-Permeable Substrate

Smoking substitute systems (e.g., e-cigarettes) are generally regarded as having fewer of the health risks associated with conventional smoking, not only for the user themselves, but also for those nearby (i.e., those affected by passive smoking). However, the exhaling of a visible vapour cloud by the user may still be regarded in some circumstances as socially unacceptable, in light of the negative views surrounding smoking itself. Therefore, many locations where smoking is not permitted also do not allow smoking substitute systems, instead requiring users to use the same designated smoking areas as smokers themselves. This can reduce the attractiveness of smoking substitute systems, and reduce their uptake as a smoking cessation aid.

Accordingly, it would be advantageous to provide a smoking substitute system that can provide a similar user experience to an e-cigarette, but without producing a visible vapour cloud. Alternatively, or additionally, it would be advantageous to provide a smoking substitute system that can allow a user to select whether or not the system produces a vapour cloud by switching between one system operating mode where a vapour cloud is produced and another system operating mode where a vapour cloud is not produced.

Still further, based on the insight of the present inventors, it would be advantageous to provide an aerosol delivery apparatus, not necessarily limited to a smoking substitute apparatus, for the delivery of an active ingredient to a user by inhalation, providing the beneficial effects referred to above.

The present disclosure (Development C) has been devised in the light of the above considerations.

According to a first aspect of Development C there is provided an aerosol delivery apparatus comprising a first reservoir region formed from an air-permeable substrate and arranged to allow air to be drawn through the first reservoir region, the first reservoir region being loaded with a source of an active ingredient and being located in a first spatial location in the aerosol delivery apparatus, and a second reservoir region formed from a substrate and arranged to allow air to be drawn in contact with the second reservoir region, the second reservoir region being loaded with a source of flavourant and being located in a second spatial location in the aerosol delivery apparatus, wherein the first and second spatial locations are not coterminous.

In some embodiments, the aerosol delivery apparatus is a smoking substitute apparatus. In such embodiments, the active ingredient may comprise or consist of nicotine.

The second reservoir region may be formed from an air-permeable substrate, so that in use air is drawn through the second reservoir region.

Optionally, the first reservoir region and the second reservoir region may be comprised within a monolithic substrate.

Conveniently, the first reservoir region may be comprised within a first substrate, and the second reservoir region may be comprised within a second substrate.

Advantageously, the first reservoir region and the second reservoir region may be arranged within the aerosol delivery apparatus such that a gap is defined between the substrate of the first reservoir region and the substrate of the second reservoir region. Accordingly, the first reservoir region and the second reservoir region may not be in contact with each other.

Optionally, the first spatial location and the second spatial location may be separated along a direction parallel to the direction of air being drawn through the first and second reservoir region.

Conveniently, the first spatial location and the second spatial location may be separated along a direction perpendicular to the direction of air being drawn through first and second reservoir region.

Advantageously, the apparatus may further comprise an air inlet and a passage leading from the air inlet to a first outlet, and the first reservoir region and the second reservoir region may be arranged within the passage.

Optionally, a heater may be arranged in the passage, the heater being configured to heat air being drawn through the first and second reservoir regions.

Conveniently, the apparatus may further comprise a one-way valve arranged along the air passage and configured to allow air to flow along the air passage in an upstream to downstream direction.

Advantageously, the first and second reservoir regions may be consumable components of the smoking substitute apparatus.

Optionally, the first and second reservoir regions may be comprised within the same consumable component of the smoking substitute apparatus.

Conveniently, the apparatus may further comprise an encapsulating material layer enclosing the first and second reservoir regions.

According to a second preferred aspect of Development C, a consumable kit of parts adapted for use in an aerosol delivery apparatus may be provided, the kit of parts comprising a first reservoir region formed from an air-permeable substrate, the first reservoir region being loaded with a source of an active ingredient, and a second reservoir region formed from a substrate, the second reservoir region being loaded with a source of flavourant wherein the first and second reservoir regions are not coterminous.

According to a third preferred aspect of Development C, an aerosol delivery apparatus may be provided that is comprised by or within a cartridge configured for engagement with a base unit, the cartridge and base unit together forming an aerosol delivery system.

According to a fourth preferred aspect of Development C, an aerosol delivery system may be provided, the system comprising a base unit, and an aerosol delivery apparatus of the third preferred aspect of Development C, and the aerosol delivery apparatus may be removably engageable with the base unit.

According to a fifth preferred aspect of Development C, a method of using an aerosol delivery apparatus of the first, second, or third aspects of Development C may be provided, the method comprising a step of generating an aerosol.

Development D: A Smoking Substitute System Wherein the Nicotine Reservoir is a Porous Polymer Loaded with Nicotine

Smoking substitute systems (e.g., e-cigarettes) are generally regarded as having fewer of the health risks associated with conventional smoking, not only for the user themselves, but also for those nearby (i.e., those affected by passive smoking). However, the exhaling of a visible vapour cloud by the user may still be regarded in some circumstances as socially unacceptable, in light of the negative views surrounding smoking itself. Therefore, many locations where smoking is not permitted also do not allow smoking substitute systems, instead requiring users to use the same designated smoking areas as smokers themselves. This can reduce the attractiveness of smoking substitute systems, and reduce their uptake as a smoking cessation aid.

Accordingly, it would be advantageous to provide a smoking substitute system that can provide a similar user experience to an e-cigarette, but without producing a visible vapour cloud. Alternatively, or additionally, it would be advantageous to provide a smoking substitute system that can allow a user to select whether or not the system produces a vapour cloud by switching between one system operating mode where a vapour cloud is produced and another system operating mode where a vapour cloud is not produced.

The present disclosure (Development D) has been devised in the light of the above considerations.

In a general aspect of Development D, the present disclosure relates to a smoking substitute system wherein the nicotine reservoir is a porous polymer loaded with nicotine, and wherein a heater is arranged upstream of the reservoir to improve nicotine entrainment in air drawn through the system.

According to a first preferred aspect of Development D there is provided a smoking substitute system comprising an air passage, a reservoir formed from an air-permeable substrate arranged in the air passage such that substantially all of the air drawn through the passage is drawn through the reservoir, the air-permeable substrate being loaded with substantially pure nicotine, and a heater arranged in the air passage and upstream of the reservoir, the heater being operable to heat air passing through the air passage.

Configuring a system in this way allows enhanced delivery of a non-visible aerosol by heating air before it is drawn through a porous reservoir, increasing the level of nicotine entrainment.

The air-permeable substrate may be formed from a polymer such as a porous foamed polymer.

Optionally, the heater for heating the air in the air passage may comprise an electrically heatable mesh.

Conveniently, the electrically heatable mesh may formed as a sheet and the sheet may be configured in a helical arrangement extending along the passage. This allows for increased heater surface area for improved heating of the air.

Advantageously, the heater for heating the air in the air passage may comprise a plurality of meshes having a common electrical connection.

Optionally, the heater for heating the air in the air passage may comprise a plurality of meshes connected in series.

Conveniently, the heater for heating the air in the air passage may comprise a plurality of meshes connected in parallel.

Advantageously, the heater for heating the air in the air passage may comprise a plurality of meshes which are independently operable.

This may allow, for example, for redundancy for failed meshes, and/or for configuring different heater profiles by controlling each of the plurality of meshes to operate for a different time period or at a different power level.

Optionally, the heater for heating the air in the air passage may comprise an electrically heatable heating coil.

Conveniently, the heater for heating the air in the air passage may comprise an electrically heatable heating plate.

Advantageously, the heating plate may surround and define a part of the air passage.

Providing a heating plate as part of the wall of the air passage allows for heating without obstructing air flow through the passage, which may be advantageous if consistent airflow is required.

Optionally, the heater for heating the air in the air passage may be heatable by resistive heating using an electrical current.

Conveniently, one or more one-way valves may be arranged along the air passage and configured to allow air to flow along the air passage in an upstream to downstream direction.

Providing one or more one-way valves arranged both upstream and downstream of an aerosol generator in the air passage can reduce, prevent or substantially prevent air flow along the air passage in a downstream to upstream direction, controlling flow of both air and vapour through the passage and apparatus.

According to a second preferred aspect of Development D, there is provided a kit of parts for a smoking substitute system according to the first aspect, comprising a base unit and a smoking substitute apparatus removably engageable with the base unit. The base unit comprises a base unit air passage, said base unit air passage comprising the heater. The smoking substitute apparatus is a cartridge configured for engagement with the base unit, the cartridge comprising a cartridge air passage in fluid communication with the base unit air passage to collectively form the air passage of the smoking substitute system, said cartridge air passage comprising the reservoir.

According to a third preferred aspect of Development D, there is provided a kit of parts for a smoking substitute system according to the first aspect of Development D comprising a base unit, and a smoking substitute apparatus removably engageable with the base unit. The smoking substitute apparatus is a cartridge configured for engagement with the base unit, the cartridge comprising at least the portion of the air passage that comprises the heater and the reservoir of the smoking substitute system.

According to a fourth preferred aspect of Development D, there is provided a method of using a smoking substitute system according to the first aspect of Development D to generate an aerosol.

Development E: An Aerosol Delivery Apparatus Comprising One-Way Valves Both Upstream and Downstream of the Aerosol Generating Part

Smoking substitute systems (e.g., e-cigarettes) are generally regarded as having fewer of the health risks associated with conventional smoking. However, condensation of the generated aerosol or vapour within the device may present a hygiene issue in long-term usage of the system.

Accordingly, it would be advantageous to provide a smoking substitute system wherein the movement of vapour within the system can be controlled so as to reduce condensation in undesired parts of the fluid passages within such a system.

Still further, based on the insight of the present inventors, it would be advantageous to provide an aerosol delivery apparatus, not necessarily limited to a smoking substitute apparatus, for the delivery of an active ingredient to a user by inhalation, providing the beneficial effects referred to above.

The present disclosure (Development E) has been devised in the light of the above considerations.

In a general aspect of Development E, the present disclosure relates to an aerosol delivery apparatus comprising one-way valves both upstream and downstream of the aerosol generating part so as to control air flow through the apparatus.

According to a first preferred aspect of Development E there is provided an aerosol delivery apparatus comprising an air passage, an aerosol generator arranged in the air passage, a first one-way valve arranged upstream of the aerosol generator along the air passage and configured to allow air to flow along the air passage in an upstream to downstream direction, and a second one-way valve arranged downstream of the aerosol generator along the air passage, and configured to allow air to flow along the air passage in an upstream to downstream direction.

Providing one one-way valves arranged both upstream and downstream of an aerosol generator in the air passage can reduce, substantially prevent or prevent air flow along the air passage in a downstream to upstream direction, controlling flow of both air and vapour through the passage and apparatus.

Optionally, a one-way valve of the one-way valves may be a duckbill valve.

Conveniently, a one-way valve of the one-way valves may be a ball one-way valve.

Advantageously, the ball one-way valve may further comprise a spring to locate the ball within the valve.

In some embodiments, the aerosol delivery apparatus is a smoking substitute apparatus. In such embodiments, the active ingredient may comprise or consist of nicotine.

Optionally, the aerosol generator may comprise a reservoir formed from an air-permeable substrate and arranged in the air passage to allow air to be drawn through the reservoir, the reservoir being loaded with a source of an active ingredient (e.g., nicotine).

The incorporation of a one-way valve may allow there to be a threshold pressure difference below which air flow will be blocked by the valve even in the upstream to downstream direction. This can ensure that the one-way valve is only opened, for example, when a user draws air through the apparatus. Such an arrangement can serve to prolong the useful lifetime of the reservoir formed from an air-permeable substrate.

Conveniently, the aerosol delivery apparatus may further comprise a heater arranged in the air passage and upstream of the reservoir, the heater being operable to heat air passing through the air passage.

Advantageously, a one-way valve of the one-way valves may be arranged upstream of the heater.

Optionally, the heater for heating the air in the air passage may comprise an electrically heatable mesh.

Conveniently, the heater for heating the air in the air passage may be heatable by resistive heating using an electrical current.

Advantageously, the aerosol generator may comprise a porous wick which, in use, wicks aerosol precursor from a reservoir to the first passage for entrainment in air flowing downstream of the aerosol generator.

Optionally, the aerosol delivery apparatus may further comprise a heater operable to generate the aerosol from the aerosol precursor, the heater being a heating filament that is wound around a portion of the porous wick.

Conveniently, the heater for generating the aerosol from the aerosol precursor may be heatable by resistive heating using an electrical current.

Advantageously, the passage may comprise a vaporisation chamber in which the aerosol generator is arranged, wherein the vaporisation chamber has a larger cross sectional diameter than a downstream part of the passage.

Optionally, the aerosol generator may comprise an aerosol-forming substrate comprising tobacco material, and a heating element operable to heat the aerosol-forming substrate to generate an aerosol for entrainment in air flowing downstream of the aerosol generator.

Conveniently, the heating element for heating the aerosol-forming substrate may comprise an electrically heatable rod.

Advantageously, the heating element for heating the aerosol-forming substrate may be heatable by resistive heating using an electrical current.

According to a second preferred aspect of Development E, there is provided an aerosol delivery apparatus according to the first aspect of Development E, wherein the aerosol delivery apparatus is comprised by or within a cartridge configured for engagement with a base unit, the cartridge and base unit together forming an aerosol delivery system.

According to a third preferred aspect of Development E, there is provided an aerosol delivery system comprising a base unit and an aerosol delivery apparatus of the second aspect, wherein the aerosol delivery apparatus is removably engageable with the base unit.

According to a fourth preferred aspect of Development E, there is provided a method of using an aerosol delivery apparatus of the first or second aspects of Development E to generate an aerosol.

In some embodiments, one or more of the fluid (air) passages may comprise a wick in fluid communication with a tank containing an aerosol precursor. The wick may comprise a porous material, capable of wicking the aerosol precursor. A portion of the wick may be exposed to air flow in the passage. The wick may also comprise one or more portions in contact with liquid stored in the reservoir. For example, opposing ends of the wick may protrude into the reservoir and an intermediate portion (between the ends) may extend across the passage so as to be exposed to air flow in the passage. Thus, liquid may be drawn (e.g., by capillary action) along the wick, from the reservoir to the portion of the wick exposed to air flow.

The wick may be heated by a heater. The heater of the wick may comprise a heating element, which may be in the form of a filament wound about the wick (e.g., the filament may extend helically about the wick in a coil configuration). The heating element may be wound about the intermediate portion of the wick that is exposed to air flow in the passage. The heating element may be electrically connected (or connectable) to a power source. Thus, in operation, the power source may apply a voltage across the heating element so as to heat the heating element by resistive heating. This may cause liquid stored in the wick (i.e., drawn from the tank) to be heated so as to form a vapour and become entrained in air flowing through the passage. This vapour may subsequently cool to form an aerosol in the passage, typically downstream from the heating element.

In an arrangement where the aerosol delivery apparatus comprises a wick arranged so as to be exposed to air flow in the passage, the aerosol delivery apparatus may comprise a vaporisation chamber. The vaporisation chamber may form part of the passage in which the heater is located. The vaporisation chamber may be arranged to be in fluid communication with the inlet and outlet of the passage. The vaporisation chamber may be an enlarged portion of the passage. In this respect, the air as drawn in by the user may entrain the generated vapour in a flow away from heater. The entrained vapour may form an aerosol in the vaporisation chamber, or it may form the aerosol further downstream along the passage. The vaporisation chamber may be at least partially defined by the tank. The tank may substantially (or fully) define the vaporisation chamber. In this respect, the tank may surround the vaporisation chamber, e.g., in an annular arrangement around the vaporisation chamber.

The disclosure includes the combination of the aspects and preferred features described except where such a combination is clearly impermissible or expressly avoided.

### SUMMARY OF THE FIGURES

So that the disclosure may be understood, and so that further developments, aspects and features thereof may be appreciated, embodiments illustrating the principles of the disclosure will now be discussed in further detail with reference to the accompanying figures, in which:

FIG. 1 is a schematic front view of a smoking substitute system, according to a first embodiment of Development A, in an engaged position;

FIG. 2 is a schematic front view of the smoking substitute system of the first embodiment of Development A in a disengaged position;

FIG. 3 is a schematic longitudinal cross sectional view of a smoking substitute apparatus of the first embodiment of Development A;

FIG. 4A is a schematic longitudinal cross sectional view of a duckbill one-way valve in a closed position;

FIG. 4B is a schematic longitudinal cross sectional view of a duckbill one-way valve in an open position;

FIG. 5A is a schematic longitudinal cross sectional view of a ball one-way valve in a closed position; and

FIG. 5B is a schematic longitudinal cross sectional view of a ball one-way valve in an open position.

FIG. 6 is a schematic front view of a smoking substitute system, according to a first embodiment of Development B, in an engaged position;

FIG. 7 is a schematic front view of the smoking substitute system of the first embodiment of Development B in a disengaged position;

FIG. 8 is a schematic longitudinal cross sectional view of a smoking substitute apparatus of the first embodiment of Development B;

FIG. 9 is a schematic longitudinal cross sectional view of a smoking substitute system having a heat source arranged in a base unit;

FIG. 10 is a schematic longitudinal cross sectional view of heat source and convoluted flow path of the first embodiment of Development B; and

FIG. 11 is a schematic longitudinal cross sectional view of a smoking substitute apparatus comprising a metallic disc and a plunger for activation of the heat source.

FIG. 12 is a schematic front view of a smoking substitute system, according to a first embodiment of Development C, in an engaged position;

FIG. 13 is a schematic front view of the smoking substitute system of the first embodiment of Development C in a disengaged position;

FIG. 14 is a schematic longitudinal cross sectional view of a reference arrangement of a smoking substitute apparatus, provided for contrast with embodiments of Development C; and

FIG. 15 is a schematic longitudinal cross sectional view of a smoking substitute apparatus of the first embodiment of Development C; and

FIGS. 16A, 16B and 16C are enlarged schematic cross sectional views of exemplary arrangements for the first and second reservoir regions of the first embodiment of Development 7C.

FIG. 17 is a schematic front view of a smoking substitute system, according to a first embodiment of Development D, in an engaged position;

FIG. 18 is a schematic front view of the smoking substitute system of the first embodiment of Development D in a disengaged position;

FIG. 19 is a schematic longitudinal cross sectional view of a smoking substitute apparatus of the first embodiment of Development D;

FIG. 20 is a schematic longitudinal cross sectional view of a heater of the smoking substitute apparatus of the first embodiment of Development D;

FIG. 21 is a schematic longitudinal cross sectional view of a heater of the smoking substitute apparatus of the first embodiment of Development D;

FIG. 22 is a schematic longitudinal cross sectional view of a heater of the smoking substitute apparatus of the first embodiment of Development D;

FIG. 23 is a schematic longitudinal cross sectional view of a heater of the smoking substitute apparatus of the first embodiment of Development D, in which the meshes are independently controllable;

FIG. 24 is a schematic longitudinal cross sectional view of a heater of the smoking substitute apparatus of the first embodiment of Development D;

FIG. 25A is a schematic longitudinal cross sectional view of a heater of the smoking substitute apparatus of the first embodiment of Development D; and

FIG. 25B is a schematic top-down view of a heater of the smoking substitute apparatus of the first embodiment of Development D.

FIG. 26 is a schematic front view of a smoking substitute system, according to a first embodiment of Development E, in an engaged position;

FIG. 27 is a schematic front view of the smoking substitute system of the first embodiment of Development E in a disengaged position;

FIG. 28 is a schematic longitudinal cross sectional view of a smoking substitute apparatus of the first embodiment of Development E;

FIG. 29 is a schematic longitudinal cross sectional view of a smoking substitute apparatus of the second embodiment of Development E;

FIG. 30 is an enlarged schematic cross sectional view of part of the air passage and vaporisation chamber of the second embodiment of Development E;

FIG. 31 is a schematic longitudinal cross sectional view of a smoking substitute apparatus of the third embodiment of Development E;

FIG. 32A is a schematic longitudinal cross sectional view of a duckbill one-way valve in a closed position;

FIG. 32B is a schematic longitudinal cross sectional view of a duckbill one-way valve in an open position;

FIG. 33A is a schematic longitudinal cross sectional view of a ball one-way valve in a closed position; and

FIG. 33B is a schematic longitudinal cross sectional view of a ball one-way valve in an open position.

### DETAILED DESCRIPTION OF THE DISCLOSURE

Further background to the present disclosure and further developments, aspects and embodiments of the present disclosure will now be discussed with reference to the accompanying figures. Further developments, aspects and embodiments will be apparent to those skilled in the art. The contents of all documents mentioned in this text are incorporated herein by reference in their entirety.

The embodiments of the disclosure are described as smoking substitute apparatuses or systems, in which the active ingredient typically comprises or consists of nicotine. However, on the basis of the present disclosure it will be apparent that the disclosure can be embodied more generally as an aerosol delivery apparatus or system. In such aerosol delivery apparatuses or systems the active ingredient may not comprise nicotine, and may instead comprise or consist of one or more of a nutritional agent, a pharmaceutical agent or a flavour agent.

Development A: An Aerosol Delivery Apparatus Comprising an Air Permeable Reservoir and at Least One-Way Valve Arranged in the Air Passage

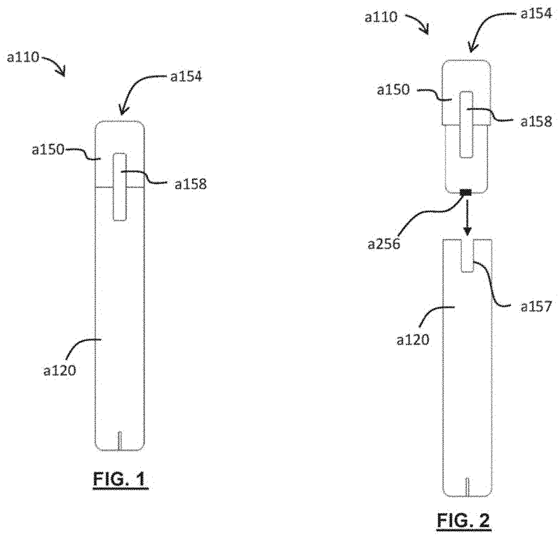

FIGS. 1 and 2 illustrate a smoking substitute system in the form of an e-cigarette system a110. The system a110 comprises a main body a120 of the system a110, and a smoking substitute apparatus in the form of an e-cigarette consumable (or “pod”) a150. In the illustrated embodiment the consumable a150 (sometimes referred to herein as a smoking substitute apparatus) is removable from the main body a120, so as to be a replaceable component of the system a110. The e-cigarette system a110 is a closed system in the sense that it is not intended that the consumable should be refillable with nicotine-based liquid by a user.

As is apparent from FIGS. 1 and 2, the consumable a150 is configured to engage the main body a120. FIG. 1 shows the main body a120 and the consumable a150 in an engaged state, whilst FIG. 2 shows the main body a120 and the consumable a150 in a disengaged state. When engaged, a portion of the consumable a150 is received in a cavity of corresponding shape in the main body a120 and is retained in the engaged position by way of a snap-engagement mechanism. In other embodiments, the main body a120 and consumable a150 may be engaged by screwing one into (or onto) the other, or through a bayonet fitting, or by way of an interference fit. In the illustrated embodiment, the consumable a150 is a “single-use” consumable a150. The term “single-use” does not necessarily mean the consumable is designed to be disposed of after a single smoking session. Rather, it defines that the consumable a150 is not designed to be refilled, and is instead disposed of and replaced after usage.

The power source of the main body a120 may be in the form of a battery (e.g., a rechargeable battery such as a lithium ion battery). The main body a120 may comprise a connector in the form of, e.g., a USB port for recharging this battery. The main body a120 may also comprise a controller that controls the supply of power from the power source to the main body electrical contacts (and thus to the heater a164). That is, the controller may be configured to control a voltage applied across the main body electrical contacts, and thus the voltage applied across the heater a164. In this way, the heater a164 may only be heated under certain conditions (e.g., during a puff and/or only when the system is in an active state). In this respect, the main body a120 may include a puff sensor (not shown) that is configured to detect a puff (i.e., inhalation). The puff sensor may be operatively connected to the controller so as to be able to provide a signal, to the controller, which is indicative of a puff state (i.e., puffing or not puffing). The puff sensor may, for example, be in the form of a pressure sensor or an acoustic sensor.

Although not shown, the main body a120 and consumable a150 may comprise a further interface which may, for example, be in the form of an RFID reader, a barcode or QR code reader. This interface may be able to identify a characteristic (e.g., a type) of a consumable a150 engaged with the main body a120. In this respect, the consumable a150 may include any one or more of an RFID chip, a barcode or QR code, or memory within which is an identifier and which can be interrogated via the interface.

The system a110 is configured to vaporise an aerosol precursor, which in the illustrated embodiment is a nicotine-based liquid impregnated into a substrate a160. When air is drawn through or over the nicotine-impregnated substrate a160, the nicotine is vaporised and entrained in the airflow to thereby be delivered to a user. The vapour or aerosol produced by the aerosol generator is less visible than that produced by a conventional e-liquid from an e-cigarette when exhaled by a user. Preferably, the vapour or aerosol generated by the aerosol generator is invisible or substantially invisible when exhaled by a user. The porosity or air permeability of at least part of the substrate a160 may be selected so as to provide a resistance to draw to a user that is comparable to a conventional cigarette.

A substrate a160 may be impregnated with nicotine by immersion in a solution of nicotine in a volatile carrier solvent (e.g., ethanol) such that the substrate a160 is evenly soaked. The substrate a160 can then be removed and left to dry or baked in an oven, meaning that the carrier is evaporated and the nicotine is left evenly spread throughout the substrate. The nicotine solution may further comprise a flavourant. Alternatively, a flavourant solution and a nicotine solution may be separately impregnated into the substrate a160.

Further details are now set out relating to the air-permeable substrate and its impregnation with an active ingredient. These further details are applicable to all described embodiments of the developments of the disclosure, unless the context demands otherwise.

Air-Permeable Substrate

Suitable materials and methods for manufacturing air permeable substrates are disclosed, for example, in U.S. Pat. No. 4,800,903, U.S. Pat. No. 4,284,089, U.S. Pat. No. 4,813,437, and U.S. Pat. No. 5,167,242, the entire contents of which are incorporated herein by reference.

U.S. Pat. No. 4,800,903 discloses that preferred materials for a polymeric plug are olefinic polymers, and preferably polyethylene or polypropylene, most preferably high density polyethylene. Use of high density polyethylene is preferred over, for example, amorphous polyethylene, since it provides a balance between ease of manufacturing and capacity for reversible nicotine absorption.

Meanwhile, some polymers are considered to be inherently unsuitable for use as an air permeable substrate. For example, some polymeric substances such as polystyrene and polycarbonate are dissolved by nicotine, rendering them unsuitable for forming a nicotine impregnated substrate. Furthermore, polymers containing halogens or nitrogen or sulphur are undesirable since they can produce noxious fumes.

To improve user satisfaction, it may be preferable to use an air permeable substrate that provides an equivalent resistance to draw to that of a conventional cigarette. For example, U.S. Pat. No. 4,284,089 discloses that a non-combustible cigarette with a draw resistance approximating that of a conventional cigarette would permit about 35 millilitres of air to be drawn through it during a 2 second period.

A substrate may be impregnated with nicotine by a variety of methods. For example, U.S. Pat. No. 4,800,903 indicates that liquid nicotine, nicotine vapour or a solution of nicotine may be used, and suggests that a solution of nicotine in supercritical liquid carbon dioxide may advantageously be used to impregnate the substrate. Alternatively, the substrate may be impregnated with nicotine via immersion in a liquid containing nicotine and a volatile carrier (for example a solution of nicotine in ethanol). The substrate is immersed to evenly soak the substrate. Once the substrate is removed from the liquid it can be left to dry or baked in an oven, evaporating away the carrier so that the nicotine is left evenly distributed throughout the substrate.

U.S. Pat. No. 5,167,242 discloses that a polyethylene plug can be charged or loaded with a mixture of nicotine, menthol and ethanol in a weight ratio (nicotine:menthol:ethanol) of about 10:1:120 or 10:1:160. The menthol and nicotine are sequentially added to the ethanol in a mixing vessel to produce a solution. Meanwhile, the plugs are placed in a vacuum dryer, which is partially evacuated to create a lower internal pressure than that of the mixing vessel, allowing the nicotine/ethanol/methanol solution to be sucked into the vacuum dryer. The plugs remain immersed in the solution within the vacuum dryer for 10 minutes, after which the temperature is raised and the vacuum pump is started to evaporate the ethanol. The vacuum dryer is then filled with nitrogen, and a nitrogen atmosphere is maintained for the remainder of the packaging procedure to prevent oxygen contamination of the nicotine.

In alternative embodiments, the air-permeable substrate may be formed in a different manner. For example, the air-permeable substrate may be formed from tobacco. The tobacco may be leaf tobacco, tobacco derivatives, expanded tobacco, shredded tobacco, reconstituted tobacco or tobacco substitutes. Preferably the tobacco has a relatively low moisture content, for example less than 10 wt % moisture. A typical minimum moisture content for the tobacco is not less than 4 wt % moisture. Such low moisture content tobacco, when exposed to heated air, would typically not produce a substantial vapour. Accordingly, such an air-permeable substrate may be loaded with a source of an active ingredient, as described above.

Where the air-permeable substrate is formed for example of tobacco, the active agent may be applied to the air-permeable substrate by mixing and/or dissolving the active agent in a suitable carrier liquid such as a solvent (e.g., water, ethanol, PG, glycerine, macrogol, caster oil, paraffin, (and derivatives thereof)).

The air is typically heated to a suitable temperature. This temperature may be at least 30° C. This is in order to promote vaporisation of the active ingredient. The temperature is typically not greater than 80° C., or typically not greater than 70° C. This is in order to promote user comfort. It may also reduce or prevent the degradation of the air-permeable substrate and/or the active ingredient.

Development A—Further Details

The nicotine-impregnated substrate a160 may be provided within a consumable a150. In such an embodiment, when the supply of nicotine in the nicotine-impregnated substrate a160 is depleted, the consumable a150 may be replaced. In other embodiments, the nicotine-impregnated substrate a160 itself may be a consumable component of the system a110. For example, the nicotine-impregnated substrate may be locatable within and removable from the system a110.

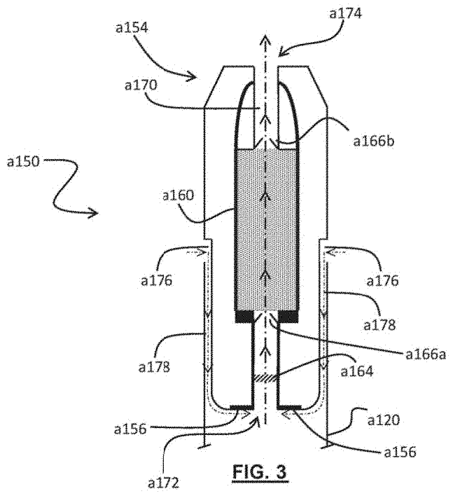

FIG. 3 shows a schematic longitudinal cross-sectional view of the aerosol generator which forms part of the smoking substitute system shown in FIGS. 1 and 2. In FIG. 3, the nicotine-impregnated substrate a160 is arranged within a passage a170. The passage a170 extends between an aerosol generator inlet a172 and an aerosol generator outlet a174 at opposing ends of the consumable a150. In this respect, the passage a170 comprises an upstream end at the end of the consumable a150 that engages with the main body a120, and a downstream end at an opposing end of the consumable a150 that comprises a mouthpiece a154 of the system a110.

When the consumable a150 is received in the cavity of the main body a120 as shown in FIG. 3, a plurality of device air inlets a176 are formed at the boundary between the casing of the consumable and the casing of the main body. The device air inlets a176 are in fluid communication with the aerosol generator inlet a172 through an inlet flow channel a178 formed in the cavity of the main body which is of corresponding shape to receive a part of the consumable a150a. Air from outside of the system a110 can therefore be drawn into the passage a170 through the device air inlets a176 and the inlet flow channels a178.

When the consumable a150 is engaged with the main body a120, a user can inhale (i.e., take a puff) via the mouthpiece a154 so as to draw air through the passage a170, and so as to form an airflow (indicated by the dashed arrows in FIG. 3) in a direction from the aerosol generator inlet a172 to the aerosol generator outlet a174. Air is thereby drawn through and/or around the nicotine-impregnated substrate a160, such that nicotine from the nicotine-impregnated substrate a160 can be entrained in the airflow.

In this embodiment, a heater a164 is arranged upstream of the nicotine-impregnated substrate a160 along the passageway a170. The heater a164 is operable to heat air passing through the passageway a170 to enable or enhance nicotine entrainment. In this embodiment, the heater a164 comprises an electrically heatable mesh located in the airflow stream. The mesh may be formed of a material that is heatable by resistive heating using an electrical current. In other embodiments, the heater a164 may comprise a plurality of meshes. The heater a164 may be located in a passage in the base unit a120 (not illustrated), with the base unit passageway being in fluid communication with the passageway a170 of the consumable a150a. In further embodiments, the heater a164 may be located externally of the passageway a170 but placed in thermal communication with the airflow via thermally conductive elements which extend into or across the passageway a170 (not illustrated). In still further embodiments, the heater may be omitted.

When the consumable a150 is engaged with the main body a120, electrical contacts a156 make contact with corresponding electrical contacts (not shown) of the main body a120. The main body electrical contacts are electrically connectable to a power source (not shown) of the main body a120, such that (in the engaged position) the heater a164 is electrically connectable to the power source. In this way, power can be supplied by the main body a120 to the heater a164 in order to heat the heater a164.

The passageway a170 may comprise one or more one-way valves a166. The one or more valves serve to control airflow through the passageway a170. The one or more valves are provided to prevent, substantially prevent or reduce airflow from a downstream to upstream direction along the passageway a170. A one-way valve may also be referred to as a check valve. In FIG. 3, two valves a166 are located immediately upstream and immediately downstream, respectively, of the nicotine-impregnated substrate a160. In some embodiments, only one valve a166 may be provided. In further embodiments, a valve a166 may be located upstream of the heater a164.

Providing valves a166 immediately upstream and/or immediately downstream of the nicotine-impregnated substrate a160 may be advantageous for preventing nicotine or nicotine infused liquid from escaping from the nicotine-impregnated substrate a160 when the smoking substitute apparatus a150 is not in use. Providing a valve a166 upstream of a heater a164 allows control over the airflow, while not requiring the valve to be suitable to operate with heated air. A valve a166 provided upstream of a heater a164 may also improve the efficiency of utilisation for the heater, since heated air is prevented from flowing in an upstream direction from the heater a164.

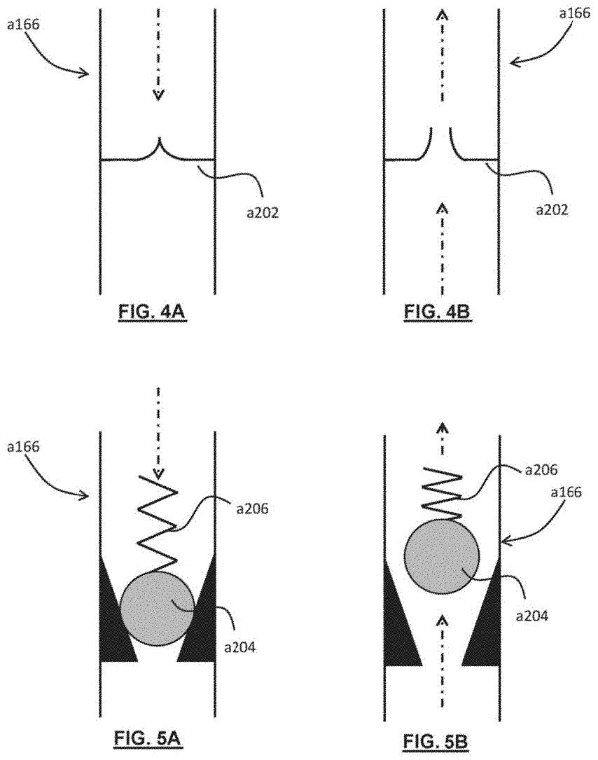

At least one of the one-way valves a166 may be a duckbill valve, as illustrated exemplarily in FIGS. 4A, 4B. A duckbill valve comprises an elastomeric material diaphragm a202 in which a portion is shaped like the bill or beak of a duck, comprising an opening or aperture therethrough. Fluid flow in the “allowed” direction opens the duckbill shaped portion (FIG. 4B), while fluid flow in a reverse direction (or absence of any fluid flow) cases the duckbill shaped portion to close (FIG. 4A). A pressure differential across the valve a166 from the upstream to downstream side may be required to open the valve and allow fluid to flow.

At least one of the one-way valves a166 may be a ball one-way valve, as illustrated exemplarily in FIGS. 5A and 5B. A ball one-way valve comprises a ball a204 located in a tapered passage so as to prevent fluid flow in a reverse direction (FIG. 5A). Fluid flow in an allowed direction moves the ball a204, allowing flow around the ball a204 (FIG. 5B). A ball one-way valve may further comprise a spring a206 to position the ball a204 within the valve. In some embodiments, the spring a206 may be omitted. As with the duckbill valve, a one-way ball valve may require a pressure differential across the valve a166 in an upstream to downstream direction to open the valve a166 and allow flow through the valve a166, particularly in valves where a spring a206 is present.

The pressure differential required to open a duckbill or ball one-way valve may therefore create a resistance to draw within the passage a170 of the smoking substitute apparatus a160. The one-way valve or valves may be configured such that, in combination with the reservoir a160, the resistance to draw of the smoking substitute apparatus as a whole is comparable to that of a conventional cigarette.

Development B: An Aerosol Delivery System with a Non-Electrical Heat Source

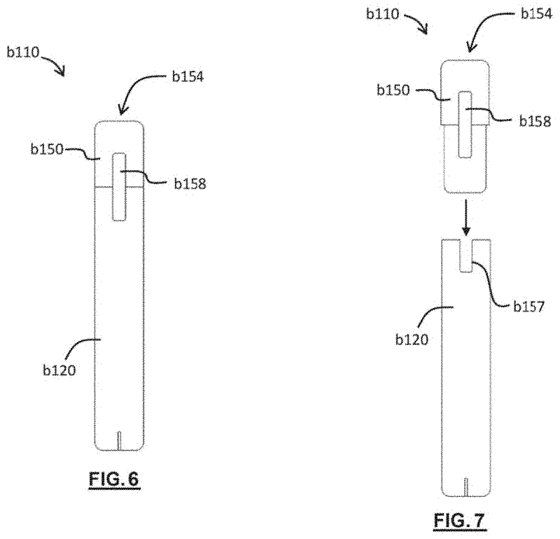

FIGS. 6 and 7 illustrate a smoking substitute system b110. The system b110 comprises a main body b120 of the system b110, and a smoking substitute apparatus in the form of a consumable (or “pod”) b150. In the illustrated embodiment the consumable b150 (sometimes referred to herein as a smoking substitute apparatus) is removable from the main body b120, so as to be a replaceable component of the system b110. The system b110 is a closed system in the sense that it is not intended that the consumable should be refillable with e-liquid by a user.

As is apparent from FIGS. 6 and 7, the consumable b150 is configured to engage the main body b120. FIG. 6 shows the main body b120 and the consumable b150 in an engaged state, whilst FIG. 7 shows the main body b120 and the consumable b150 in a disengaged state. When engaged, a portion of the consumable b150 is received in a cavity of corresponding shape in the main body b120 and is retained in the engaged position by way of a snap-engagement mechanism. In other embodiments, the main body b120 and consumable b150 may be engaged by screwing one into (or onto) the other, or through a bayonet fitting, or by way of an interference fit. In the illustrated embodiment, the consumable b150 is a “single-use” consumable b150. The term “single-use” does not necessarily mean the consumable is designed to be disposed of after a single smoking session. Rather, it defines that the consumable b150 is not designed to be refilled, and is instead disposed of and replaced after usage.

The system b110 is configured to vaporise an aerosol precursor, which in the illustrated embodiment is a nicotine-based liquid impregnated into a substrate b160. The nicotine-impregnated substrate b160 may be referred to as an aerosol generator. When air is drawn through or over the nicotine-impregnated substrate b160, the nicotine is vaporised and entrained in the airflow to thereby be delivered to a user. The vapour or aerosol produced by the aerosol generator is less visible than that produced by a conventional e-liquid from an e-cigarette when exhaled by a user. Preferably, the vapour or aerosol generated by the aerosol generator is invisible or substantially invisible when exhaled by a user. In the illustrated embodiment, the main body provides a handle by which a user may hold the system b110, but provides no functional contribution to the vaporisation of the aerosol precursor.

A substrate b160 may be impregnated with nicotine by immersion in a solution of nicotine in a volatile carrier solvent (e.g., ethanol) such that the substrate b160 is evenly soaked. The substrate b160 can then be removed and left to dry or baked in an oven, meaning that the carrier is evaporated and the nicotine is left evenly spread throughout the substrate.

The nicotine-impregnated substrate b160 may be provided within a consumable b150. In such an embodiment, when the supply of nicotine in the nicotine-impregnated substrate b160 is depleted, the consumable b150 may be replaced. In other embodiments, the nicotine-impregnated substrate b160 itself may be a consumable component of the system b110. For example, the nicotine-impregnated substrate may be locatable within and removable from the system b110.

Further details are set out above relating to the air-permeable substrate and its impregnation with an active ingredient under the heading “air-permeable substrate”, those details applying to the embodiments of Development B described here.

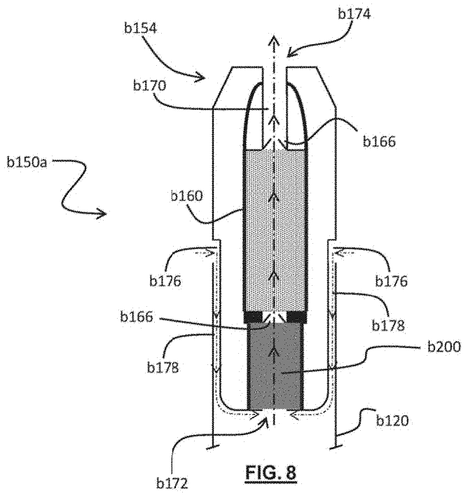

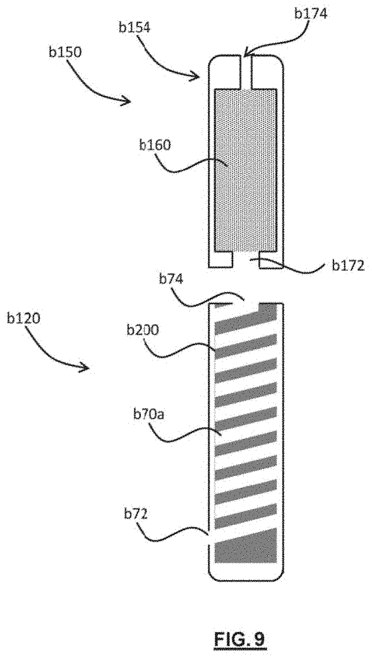

FIG. 8 shows a schematic longitudinal cross-sectional view of the aerosol generator which forms part of the substitute smoking system shown in FIGS. 6 and 7. In FIG. 8, the nicotine-impregnated substrate b160 is arranged within a passage b170. The passage b170 extends between an aerosol generator inlet b172 and an aerosol generator outlet b174 at opposing ends of the consumable b150. In this respect, the passage b170 comprises an upstream end at the end of the consumable b150 that engages with the main body b120, and a downstream end at an opposing end of the consumable b150 that comprises a mouthpiece b154 of the system b110.

When the consumable b150 is received in the cavity of the main body b120 as shown in FIG. 8, a plurality of device air inlets b176 are formed at the boundary between the casing of the consumable and the casing of the main body. The device air inlets b176 are in fluid communication with the aerosol generator inlet b172 through an inlet flow channel b178 formed in the cavity of the main body which is of corresponding shape to receive a part of the consumable b150. Air from outside of the system b110 can therefore be drawn into the passage b170 through the device air inlets b176 and the inlet flow channels b178.

When the consumable b150 is engaged with the main body b120, a user can inhale (i.e., take a puff) via the mouthpiece b154 so as to draw air through the passage b170, and so as to form an airflow (indicated by the dashed arrows in FIG. 8) in a direction from the aerosol generator inlet b172 to the aerosol generator outlet b174. Air is thereby drawn through and/or around the nicotine-impregnated substrate b160, such that nicotine from the nicotine-impregnated substrate b160 can be entrained in the airflow. The nicotine-impregnated substrate b160 is arranged to extend across a cross-section of at least a portion of the passage b170, such that substantially all of the air drawn through the passage b170 passes through at least a part of the reservoir b160. The resistance to drawing air through the consumable b150 may be configured by altering the air permeability of the nicotine-impregnated substrate, or by the provision of one or more bypass passages separate from the passage b170 (not illustrated).

A non-electrical heat source b200 is arranged upstream of the nicotine-impregnated substrate b160. The non-electrical heat source b200 generates heat to heat the air in the passage b170a by an exothermic reaction. The heat source b200 may be a replaceable component of the smoking substitute apparatus b150. The smoking substitute apparatus b150 may therefore define a receptacle or space into which a replacement non-electrical heat source b200 may be placed once the reaction has completed. Alternatively, the consumable b150 may comprise one or more non-replaceable reaction chambers, in which case the entire consumable b150 is intended to be replaced. In some embodiments, a receptacle for the non-electrical heat source b200 may be provided in the main body b120. In such an embodiment, the main body b120 further comprises a main body passage b70, having a main body inlet b72 and a main body outlet b74 in fluid communication with the inlet b172 of passage b170 of the consumable b150, the main body passage b70 following a path through or around the non-electrical heat source b200, as illustrated in FIG. 9.

The passage section b170a, between the inlet b172 and the nicotine impregnated substrate b160, which may be referred to as the heat source air passage b170a, is configured to follow a convoluted (or at least non-linear) path through or around the heat source b200. The distance travelled by air along the passage section b170a (which may be referred to as the path length of the passage section b170a) is therefore greater than the separation distance (measured along a straight line) between the inlet of the passage section b170a and the outlet of the passage section b170a on the heat source b200.

A greater path length to separation distance may be advantageous for improved heat transfer between the heat source b200 and the air in the passage section b170a. For example, the path length may be greater than 1.1 times the separation distance, preferably greater than 1.5 times the separation distance, greater than 2 times the separation distance, greater than 5 times the separation distance or greater than 10 times the separation distance. Increasing the path length may require the passage section b170a to be narrowed, and may therefore create a higher than desirable resistance to draw for the smoking substitute apparatus. Therefore, the path length may be less than 100 times the separation distance, preferably less than 80 times the separation distance, or less than 50 times the separation distance, or less than 20 times the separation distance.

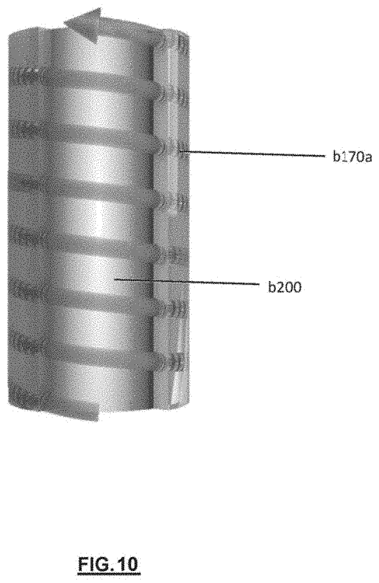

This relationship between path length and separation distance for the passage section b170a may be realised through different passage section b170a configurations. For example, the heat source b200 may be cylindrical, and the passage section b170a may be shaped to form a helical path around or through the heat source b200, as illustrated in FIG. 10. In some embodiments, the passage section b170a may be divided into a plurality of separate (i.e., unconnected) helical passage sections b170a around the heat source b200. The cross-sectional area of the convoluted section of passage b170a may vary along its length. The cross-sectional shape of the convoluted section of passage b170a may be, for example, circular, elliptical, or semi-circular.

The passage section b170a may be formed as part of the body of the smoking substitute apparatus b150. For example, the passage section b170a may be defined within the wall of the receptacle or the reaction chamber for the non-electrical heat source b200. Alternatively, a replaceable non-electrical heat source b200 may comprise a container or case for a reaction chamber in which the exothermic reaction takes place, and this case may define the passage section b170a. The passage section may be moulded into the body of the smoking substitute apparatus or a container or case for the non-electrical heat source b200.

The heat source b200 is operable to generate heat utilising an exothermic reaction. Such an exothermic reaction may be, for example, a crystallisation of a super-saturated solution. Suitable exothermic reactions include, for example, crystallisation of an aqueous solution of sodium acetate. Such reactions may be reversible, for example by heating of the solution to restore the super-saturated state. In some embodiments, therefore, the heat source b200 may be reusable.

Alternatively, a reaction between two or more chemicals may provide an exothermic reaction. For example, a reaction between lime and aluminium, between calcium oxide and water, between aluminium and silicon dioxide may be utilised. In still further embodiments, a reaction between iron powder and atmospheric oxygen may be employed, optionally in the presence of activated charcoal, vermiculite, and/or sodium chloride. Such reactions are, in practical use of the present embodiments, non-reversible, and a heat source b200 of this type is therefore consumable.

A user of the smoking substitute apparatus b150 may operate the heat source b200 by initiating the exothermic reaction. In some embodiments, the user may initiate the exothermic reaction prior to inserting the heat source b200 into the receptacle of the smoking substitute apparatus. The user may then wait until a defined time period has passed before drawing air through the smoking substitute apparatus b150, allowing the heat source b200 to reach a desired temperature. The smoking substitute apparatus b150 or the heat source b200 may comprise a means for indicating to the user that a suitable temperature has been reached. For example, an indicator may be provided that changes colour in accordance with the temperature of the heat source b200. Such an indicator may be a non-electrical means for indicating temperature, and may be, for example, a thermochromic element or a thermochromic coating on an exterior surface of the heat source b200 or the smoking substitute apparatus b150.

A user of the smoking substitute apparatus b150 may activate or commence the exothermic reaction by various processes, according to the type of reaction utilised. A crystallisation reaction may be activated using a convex or concave metallic disc b210 situated inside the chemical container. The metallic disc b210 is inverted (i.e., flexed from concave to convex) to initiate the crystallisation reaction. More generally, the crystallisation reaction may be initiated by operation of a multistable (e.g., bistable) trigger element, one example of which is a concave-convex metallic disc mentioned above. In some embodiments, the disc b210 may be inverted by the user pressing or bending the disc. In other embodiments, the disc may be inverted by the use of a plunger or piston b212, as illustrated in FIG. 11. Where two components are required to mix, this may be activated by piercing or removing a separator between the containers of the respective components. Where a reaction such as oxidation of iron powder by atmospheric oxygen is used, a tab, lid, or seal may be removed or pierced to allow air to react with the iron powder.

The passageway b170 may comprise one or more one-way valves b166. The one or more valves are provided to prevent, substantially prevent or reduce airflow from a downstream to upstream direction along the passageway b170. In FIG. 8, two valves b166 are located immediately upstream and immediately downstream, respectively, of the nicotine-impregnated substrate b160. In some embodiments, only one valve b166 may be provided. In further embodiments, a valve b166 may be located upstream of the heater b164. In still further embodiments, the valves may be omitted.

Development C: An Aerosol Delivery Apparatus Comprising a First Reservoir Region Formed from an Air-Permeable Substrate

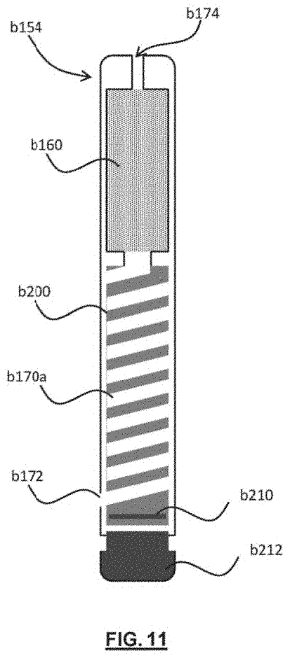

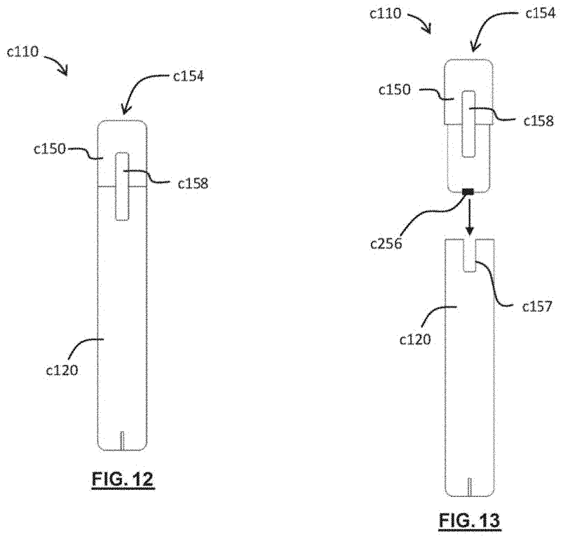

FIGS. 12 and 13 illustrate a smoking substitute system in the form of an e-cigarette system c110. The system c110 comprises a main body c120 of the system c110, and a smoking substitute apparatus in the form of an e-cigarette consumable (or “pod”) c150. In the illustrated embodiment the consumable c150 (sometimes referred to herein as a smoking substitute apparatus) is removable from the main body c120, so as to be a replaceable component of the system c110. The e-cigarette system c110 is a closed system in the sense that it is not intended that the consumable should be refillable with e-liquid by a user.

As is apparent from FIGS. 12 and 13, the consumable c150 is configured to engage the main body c120. FIG. 12 shows the main body c120 and the consumable c150 in an engaged state, whilst FIG. 12 shows the main body c120 and the consumable c150 in a disengaged state. When engaged, a portion of the consumable c150 is received in a cavity of corresponding shape in the main body c120 and is retained in the engaged position by way of a snap-engagement mechanism. In other embodiments, the main body c120 and consumable c150 may be engaged by screwing one into (or onto) the other, or through a bayonet fitting, or by way of an interference fit. In the illustrated embodiment, the consumable c150 is a “single-use” consumable c150. The term “single-use” does not necessarily mean the consumable is designed to be disposed of after a single smoking session. Rather, it defines that the consumable c150 is not designed to be refilled, and is instead disposed of and replaced after usage.

The power source of the main body c120 may be in the form of a battery (e.g., a rechargeable battery such as a lithium ion battery). The main body c120 may comprise a connector in the form of, e.g., a USB port for recharging this battery. The main body c120 may also comprise a controller that controls the supply of power from the power source to the main body electrical contacts (and thus to the filament c264 or heater c164). That is, the controller may be configured to control a voltage applied across the main body electrical contacts, and thus the voltage applied across the heater c164 or filament c264. In this way, the heater c164 or filament c264 may only be heated under certain conditions (e.g., during a puff and/or only when the system is in an active state). In this respect, the main body c120 may include a puff sensor (not shown) that is configured to detect a puff (i.e., inhalation). The puff sensor may be operatively connected to the controller so as to be able to provide a signal, to the controller, which is indicative of a puff state (i.e., puffing or not puffing). The puff sensor may, for example, be in the form of a pressure sensor or an acoustic sensor.

Although not shown, the main body c120 and consumable c150 may comprise a further interface which may, for example, be in the form of an RFID reader, a barcode or QR code reader. This interface may be able to identify a characteristic (e.g., a type) of a consumable c150 engaged with the main body c120. In this respect, the consumable c150 may include any one or more of an RFID chip, a barcode or QR code, or memory within which is an identifier and which can be interrogated via the interface.

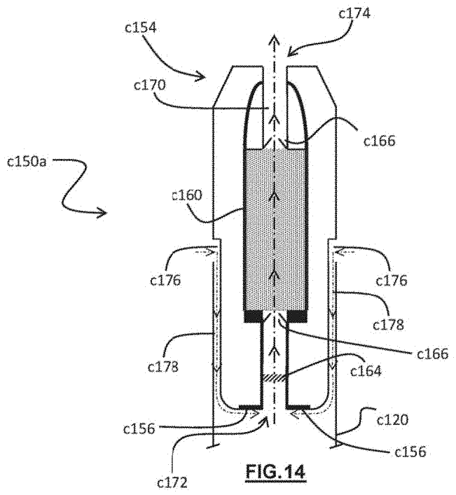

The system c110 is configured to vaporise an aerosol precursor, which in the reference arrangement illustrated in FIG. 14 comprises a nicotine-based liquid impregnated into a substrate c160. The nicotine-impregnated substrate c160 may be referred to as a reservoir. When air is drawn through or over the nicotine-impregnated substrate c160, the nicotine is vaporised and entrained in the airflow to thereby be delivered to a user. The vapour or aerosol produced by the aerosol generator is less visible than that produced by a conventional e-liquid from an e-cigarette when exhaled by a user. Preferably, the vapour or aerosol generated by the aerosol generator is invisible or substantially invisible when exhaled by a user. The porosity or air permeability of at least part of the substrate c160 may be selected so as to provide a resistance to draw to a user that is comparable to a conventional cigarette.

The substrate c160 may be impregnated with nicotine by immersion in a solution of nicotine in a volatile carrier solvent (e.g., ethanol) such that the substrate c160 is evenly soaked. The substrate c160 can then be removed and left to dry or baked in an oven, meaning that the carrier is evaporated and the nicotine is left evenly spread throughout the substrate.

The nicotine-impregnated substrate c160 may be provided within a consumable c150a. In such an arrangement, when the supply of nicotine in the nicotine-impregnated substrate c160 is depleted, the consumable c150a may be replaced. In other embodiments, the nicotine-impregnated substrate c160 itself may be a consumable component of the system c110. For example, the nicotine-impregnated substrate may be locatable within and removable from the system c110. A replaceable nicotine-impregnated substrate may be provided within a wrapping such as a foil or plastic wrapping material to prevent the impregnated nicotine from escaping prior to usage. The smoking substitute apparatus c150 may comprise piercing elements to pierce this wrapping. Alternatively, the wrapping may comprise seals that a user can remove prior to insertion into the smoking substitute apparatus c150.

Further details are set out above relating to the air-permeable substrate and its impregnation with an active ingredient under the heading “air-permeable substrate”, those details applying to the embodiments of Development C described here.

FIG. 14 shows a schematic longitudinal cross-sectional view of a reference arrangement for a smoking substitute apparatus which forms part of the smoking substitute system shown in FIGS. 12 and 13. In FIG. 14, the nicotine-impregnated substrate c160 is arranged within a passage c170. The passage c170 extends between an aerosol generator inlet c172 and an aerosol generator outlet c174 at opposing ends of the consumable c150. In this respect, the passage c170 comprises an upstream end at the end of the consumable c150 that engages with the main body c120, and a downstream end at an opposing end of the consumable c150 that comprises a mouthpiece c154 of the system c110.

When the consumable c150a is received in the cavity of the main body c120 as shown in FIG. 14, a plurality of device air inlets c176 are formed at the boundary between the casing of the consumable and the casing of the main body. The device air inlets c176 are in fluid communication with the aerosol generator inlet c172 through an inlet flow channel c178 formed in the cavity of the main body which is of corresponding shape to receive a part of the consumable c150a. Air from outside of the system c110 can therefore be drawn into the passage c170 through the device air inlets c176 and the inlet flow channels c178.

When the consumable c150a is engaged with the main body c120, a user can inhale (i.e., take a puff) via the mouthpiece c154 so as to draw air through the passage c170, and so as to form an airflow (indicated by the dashed arrows in FIG. 14) in a direction from the aerosol generator inlet c172 to the aerosol generator outlet c174. Air is thereby drawn through and/or around the nicotine-impregnated substrate c160, such that nicotine from the nicotine-impregnated substrate c160 can be entrained in the airflow. The nicotine-impregnated substrate c160 is arranged to extend across a cross-section of at least a portion of the passage c170, such that substantially all of the air drawn through the passage c170 passes through at least a part of the reservoir c160. The resistance to drawing air through the consumable c150a may be configured by altering the air permeability of the nicotine-impregnated substrate, or by the provision of one or more bypass passages separate from the passage c170 (not illustrated).

In this reference arrangement, a heater c164 is arranged upstream of the nicotine-impregnated substrate c160 along the passageway c170. The heater c164 is operable to heat air passing through the passageway c170 to enable or enhance nicotine entrainment. In this embodiment, the heater c164 comprises an electrically heatable mesh located in the airflow stream. The mesh may be formed of a material that is heatable by resistive heating using an electrical current. In other embodiments, the heater c164 may comprise a plurality of meshes. The heater c164 may be located in a passage in the base unit c120 (not illustrated), with the base unit passageway being in fluid communication with the passageway c170 of the consumable c150a. In further embodiments, the heater c164 may be located externally of the passageway c170 but placed in thermal communication with the airflow via thermally conductive elements which extend into or across the passageway c170 (not illustrated). In still further embodiments, the heater may be omitted.

When the consumable c150a is engaged with the main body c120, electrical contacts c156 make contact with corresponding electrical contacts (not shown) of the main body c120. The main body electrical contacts are electrically connectable to a power source (not shown) of the main body c120, such that (in the engaged position) the heater c164 is electrically connectable to the power source. In this way, power can be supplied by the main body c120 to the heater c164 in order to heat the heater c164.

The passageway c170 may comprise one or more one-way valves c166. The one or more valves are provided to prevent, substantially prevent or reduce airflow from a downstream to upstream direction along the passageway c170. In FIG. 14, two valves c166 are located immediately upstream and immediately downstream, respectively, of the nicotine-impregnated substrate c160. In some embodiments, only one valve c166 may be provided. In further embodiments, a valve c166 may be located upstream of the heater c164. In still further embodiments, the valves may be omitted.

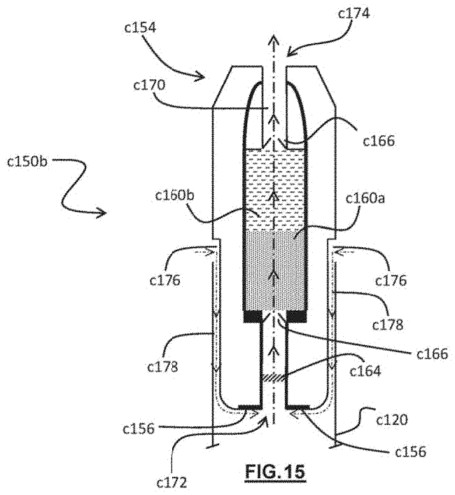

FIG. 15 shows a schematic longitudinal cross-sectional view of a first embodiment of a smoking substitute apparatus c150b. The embodiment in FIG. 15 differs from the reference arrangement in FIG. 14 in that the substrate c160 comprises two distinct reservoir regions c160a, c160b. The two reservoir regions c160a, c160b are not coterminous. In other words, the first reservoir region c160a and the second reservoir region c160b are arranged at respective first and second spatial locations within the smoking substitute apparatus c150b, wherein the first and second spatial locations are not coterminous. The first and second reservoir regions c160a, c160b may join at an interface. The first and second reservoir regions c160a, c160b may partially overlap, due, for example, to diffusion of the impregnating material within the substrate material. However, neither of the first and second reservoir regions c160a, c160b is fully comprised by the other, and there is therefore a region of the reservoir that a uniquely part of the first reservoir region c160a, and a region of the reservoir that is uniquely part of the second reservoir region c160b.

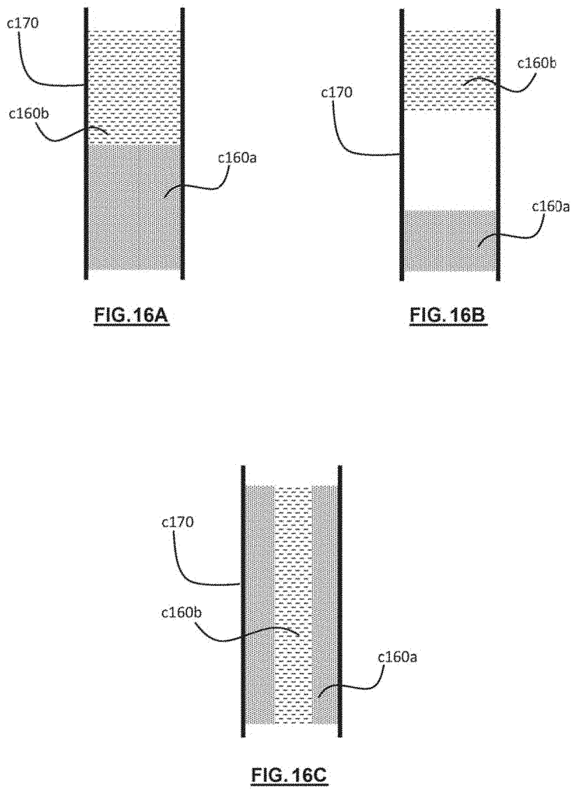

The two reservoir regions c160a, c160b may be different regions of a single, monolithic, air-permeable substrate. In other embodiments, each reservoir region may be comprised in a separate air-permeable substrate, which, in use, may be arranged adjacent to each other, and may or may not be in contact with each other. Example arrangements for the reservoir regions are illustrated in FIGS. 16A-16C. The first reservoir region c160a and second reservoir region c160b may also be reversed in position from those indicated in FIGS. 16A-16C. In other words, the second reservoir region c160b may be arranged upstream of the first reservoir region c160a, or may be arranged radially outwards of the first reservoir region c160a. In embodiments where the first reservoir region c160a and second reservoir region c160b are comprised within separate substrates, both substrates may be comprised together within a wrapping material, or may be comprised within separate wrapping materials.

As with the nicotine-impregnated substrate c160 of the reference arrangement, the first reservoir region c160a and second reservoir region c160b are arranged to extend across a cross-section of the passage c170, such that substantially all of the air drawn through the passage c170 passes through the at least one of the first reservoir region c160a and the second reservoir region c160b.

The first reservoir region c160a comprises a source of nicotine. As with the reference arrangement described above, the nicotine may be introduced into the substrate region c160a by immersing the first reservoir region c160a of a substrate in a solution of nicotine in a volatile carrier solvent (e.g., ethanol) such that the substrate c160 is evenly soaked. The substrate c160 can then be removed and left to dry or baked in an oven, meaning that the carrier is evaporated and the nicotine is left evenly spread throughout the first reservoir region.

The second reservoir region c160b comprises a source of flavourant. This flavourant may be introduced into the second reservoir region c160b according to a similar process as that for introducing nicotine into the first reservoir region c160a.

In embodiments where the first reservoir region c160a and second reservoir region c160b are comprised within a single monolithic air-permeable substrate, the first reservoir region c160a and second reservoir region c160b may be, for example, arranged at opposite sides or ends of the air permeable substrate. Each side or end may be immersed in turn to impregnate the desired nicotine and flavourant sources. In embodiments where the first reservoir region c160a and second reservoir region c160b are comprised within separate substrates, each substrate may be immersed in a respective nicotine or flavourant solution.

Comprising the first reservoir region c160a and second reservoir region c160b in a monolithic substrate is advantageous for ensuring consistency of operation within a smoking substitute apparatus, since this avoids any variations arising from relative positioning of two or more separate components. Meanwhile, comprising the first reservoir region c160a and second reservoir region c160b in separate substrates may allow for simpler manufacture, in particular since separate solutions can be impregnated without the possibility of interaction. It may also be possible to use different substrate materials for the first reservoir region c160a and second reservoir region c160b, as may be required according to solvent compatibility, or to provide different physical characteristics such as porosity. Furthermore, if the reservoir regions are comprised within different substrates, different first reservoir regions c160a and second reservoir regions c160b can be paired together. For example, a group of first reservoir regions c160a may be provided which comprise different nicotine concentrations. A second group of second reservoir regions c160b may be provided which comprise different flavourants. A first reservoir region c160a may be selected from the first group and a second reservoir region c160b may be selected from the second group to provide a desired nicotine strength and flavourant combination.

In a still further embodiment, the second reservoir region may be embodied by a flavour card. A flavour card may be a piece of card (cardboard) impregnated with a flavour.

Development D: A Smoking Substitute System Wherein the Nicotine Reservoir is a Porous Polymer Loaded with Nicotine

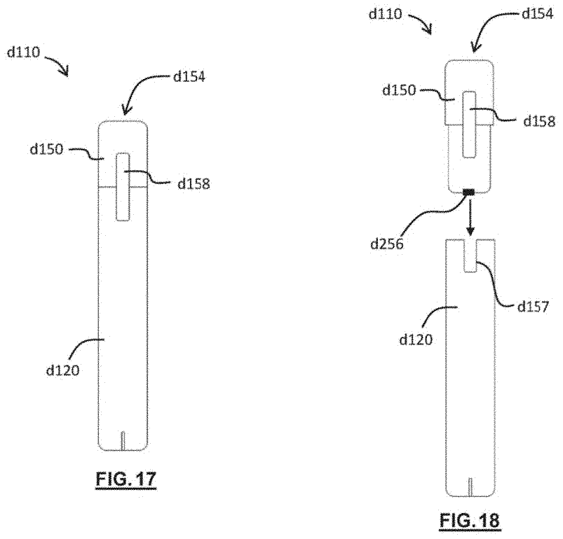

FIGS. 17 and 18 illustrate a smoking substitute system in the form of an e-cigarette system d110. The system d110 comprises a main body d120 of the system d110, and a smoking substitute apparatus in the form of an e-cigarette consumable (or “pod”) d150. In the illustrated embodiment the consumable d150 (sometimes referred to herein as a smoking substitute apparatus) is removable from the main body d120, so as to be a replaceable component of the system d110. The e-cigarette system d110 is a closed system in the sense that it is not intended that the consumable should be refillable with e-liquid by a user.

As is apparent from FIGS. 17 and 18, the consumable d150 is configured to engage the main body d120. FIG. 17 shows the main body d120 and the consumable d150 in an engaged state, whilst FIG. 18 shows the main body d120 and the consumable d150 in a disengaged state. When engaged, a portion of the consumable d150 is received in a cavity of corresponding shape in the main body d120 and is retained in the engaged position by way of a snap-engagement mechanism. In other embodiments, the main body d120 and consumable d150 may be engaged by screwing one into (or onto) the other, or through a bayonet fitting, or by way of an interference fit. In the illustrated embodiment, the consumable d150 is a “single-use” consumable d150. The term “single-use” does not necessarily mean the consumable is designed to be disposed of after a single smoking session. Rather, it defines that the consumable d150 is not designed to be refilled, and is instead disposed of and replaced after usage.

The power source of the main body d120 may be in the form of a battery (e.g., a rechargeable battery such as a lithium ion battery). The main body d120 may comprise a connector in the form of, e.g., a USB port for recharging this battery. The main body d120 may also comprise a controller that controls the supply of power from the power source to the main body electrical contacts (and thus to the heater d164). That is, the controller may be configured to control a voltage applied across the main body electrical contacts, and thus the voltage applied across the heater d164. In this way, the heater d164 may only be heated under certain conditions (e.g., during a puff and/or only when the system is in an active state). In this respect, the main body d120 may include a puff sensor (not shown) that is configured to detect a puff (i.e., inhalation). The puff sensor may be operatively connected to the controller so as to be able to provide a signal, to the controller, which is indicative of a puff state (i.e., puffing or not puffing). The puff sensor may, for example, be in the form of a pressure sensor or an acoustic sensor.

Although not shown, the main body d120 and consumable d150 may comprise a further interface which may, for example, be in the form of an RFID reader, a barcode or QR code reader. This interface may be able to identify a characteristic (e.g., a type) of a consumable d150 engaged with the main body d120. In this respect, the consumable d150 may include any one or more of an RFID chip, a barcode or QR code, or memory within which is an identifier and which can be interrogated via the interface.

The system d110 is configured to vaporise an aerosol precursor, which in the illustrated embodiment is a nicotine-based liquid impregnated into a substrate d160. The nicotine-impregnated substrate d160 may be referred to as an aerosol generator. When air is drawn through or over the nicotine-impregnated substrate d160, the nicotine is vaporised and entrained in the airflow to thereby be delivered to a user. The vapour or aerosol produced by the aerosol generator is less visible than that produced by a conventional e-liquid from an e-cigarette when exhaled by a user. Preferably, the vapour or aerosol generated by the aerosol generator is invisible or substantially invisible when exhaled by a user.

A substrate d160 may be impregnated with nicotine by immersion in a solution of nicotine in a volatile carrier solvent (e.g., ethanol) such that the substrate d160 is evenly soaked. The substrate d160 can then be removed and left to dry or baked in an oven, meaning that the carrier is evaporated and the nicotine is left evenly spread throughout the substrate.

The nicotine-impregnated substrate d160 may be provided within a consumable d150. In such an embodiment, when the supply of nicotine in the nicotine-impregnated substrate d160 is depleted, the consumable d150 may be replaced. In other embodiments, the nicotine-impregnated substrate d160 itself may be a consumable component of the system d110. For example, the nicotine-impregnated substrate may be locatable within and removable from the system d110.

Further details are set out above relating to the air-permeable substrate and its impregnation with an active ingredient under the heading “air-permeable substrate”, those details applying to the embodiments of Development D described here.

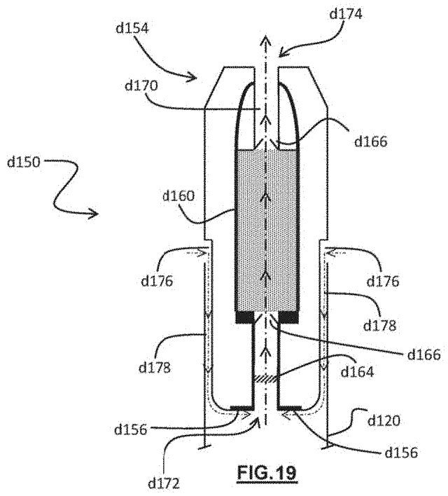

FIG. 19 shows a schematic longitudinal cross-sectional view of the aerosol generator which forms part of the smoking substitute system shown in FIGS. 17 and 18. In FIG. 19, the nicotine-impregnated substrate d160 is arranged within a passage d170. The passage d170 extends between an aerosol generator inlet d172 and an aerosol generator outlet d174 at opposing ends of the consumable d150. In this respect, the passage d170 comprises an upstream end at the end of the consumable d150 that engages with the main body d120, and a downstream end at an opposing end of the consumable d150 that comprises a mouthpiece d154 of the system d110.

When the consumable d150 is received in the cavity of the main body d120 as shown in FIG. 19, a plurality of device air inlets d176 are formed at the boundary between the casing of the consumable and the casing of the main body. The device air inlets d176 are in fluid communication with the aerosol generator inlet d172 through an inlet flow channel d178 formed in the cavity of the main body which is of corresponding shape to receive a part of the consumable d150. Air from outside of the system d110 can therefore be drawn into the passage d170 through the device air inlets d176 and the inlet flow channels d178.

When the consumable d150 is engaged with the main body d120, a user can inhale (i.e., take a puff) via the mouthpiece d154 so as to draw air through the passage d170, and so as to form an airflow (indicated by the dashed arrows in FIG. 19) in a direction from the aerosol generator inlet d172 to the aerosol generator outlet d174. Air is thereby drawn through and/or around the nicotine-impregnated substrate d160, such that nicotine from the nicotine-impregnated substrate d160 can be entrained in the airflow. The nicotine-impregnated substrate d160 is arranged to extend across a cross-section of at least a portion of the passage d170, such that substantially all of the air drawn through the passage d170 passes through at least a part of the reservoir d160. The resistance to drawing air through the consumable d150 may be configured by altering the air permeability of the nicotine-impregnated substrate, or by the provision of one or more bypass passages separate from the passage d170 (not illustrated).

In this embodiment, a heater d164 is arranged upstream of the nicotine-impregnated substrate d160 along the passageway d170. The heater d164 is operable to heat air passing through the passageway d170 to enable or enhance nicotine entrainment. The heater d164 may be located in a passage in the base unit d120 (not illustrated), with the base unit passageway being in fluid communication with the passageway d170 of the consumable d150. In further embodiments, the heater d164 may be located externally of the passageway d170 but placed in thermal communication with the airflow via thermally conductive elements which extend into or across the passageway d170 (not illustrated).

The heater d164 comprises an electrically heatable mesh located in the airflow stream. The mesh may be formed of a material that is heatable by resistive heating using an electrical current. For example, the mesh may be a woven net of wire, or punched or lasered metal sheet arranged to be electrically connected at either side or end. Suitable wires may be, for example, 0.0125 mm in diameter, while a suitable metal sheet may have a thickness of between 0.1 and 0.3 mm. A mesh d164 according to the present disclosure may have a variety of different hole shapes formed in the mesh, including, for example, circular, hexagonal, triangular, rectangular, elliptical, or square. The mesh d164 may be formed from, for example, nichrome, stainless steel, titanium or another protectively coated metal. A current used to heat the heater d164 may be, for example, between 0.5 A and 2.0 A. The heater d164 may have a resistance of between 0.6 Ω and 6.0 Ω. The heater may be operable to heat to between 200° C. and 400° C.

In alternative embodiments (not illustrated), the heater d164 may be a heating plate or heating coil arranged in or around the periphery of the air passage d170. In this way, the heater d164 may surround and define a part of the air passage d170.

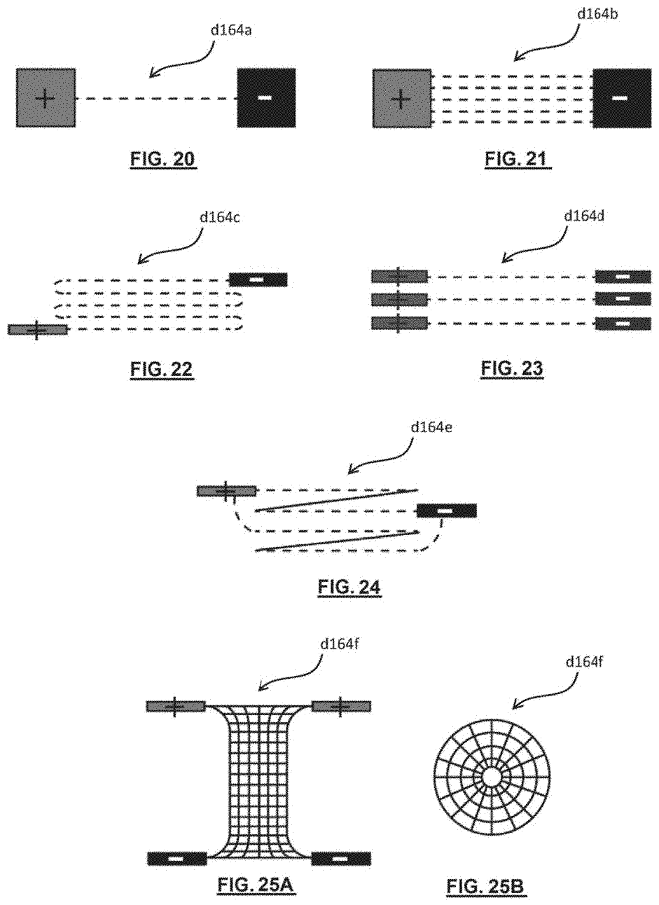

FIGS. 20-25 show exemplary arrangements for the mesh heater d164. The heater may comprise a single flat sheet mesh d164a (FIG. 20). The heater may comprise multiple sheets d164b with common connections (FIG. 21), multiple sheets d164c spaced apart and connected in series (FIG. 22), multiple sheets d164d spaced apart and connected in parallel (FIG. 23), or multiple sets of sheets d164e connected in series within a set, and in parallel between sets (FIG. 24). The spacing between the multiple sheets may be, for example, between 0.5 mm and 5 mm. One or more sheets may be arranged in a shape that extends along the passageway d170, such as a cylindrical arrangement d164f (FIG. 25A—side view, 25B—top view), or helical arrangement (not illustrated). The mesh may extend across the full cross-section of the passageway d170, or a part thereof.

The smoking substitute apparatus d150 may be configurable to heat only a subset of the mesh sheets d164. For example, a user of the smoking substitute apparatus d150 may select a number or proportion of the sheets d164 to heat according to their desired heating level. Additionally or alternatively, the smoking substitute apparatus d150 may comprise means for detecting whether one or more of the meshes d164 has become damaged such that it no longer conducts electricity, or no longer satisfactorily generates heat by resistive heating, and can thereby vary the power supplied to the remaining meshes accordingly. This may be required in particular where the heater d164 is provided as a part of the base unit d120 of the smoking substitute system d110, since it provides a redundancy for long-term deterioration of the heater d164.

When the consumable d150 is engaged with the main body d120, electrical contacts d156 make contact with corresponding electrical contacts (not shown) of the main body d120. The main body electrical contacts are electrically connectable to a power source (not shown) of the main body d120, such that (in the engaged position) the heater d164 is electrically connectable to the power source. In this way, power can be supplied by the main body d120 to the heater d164 in order to heat the heater d164.

The passageway d170 may comprise one or more one-way valves d166. The one or more valves are provided to prevent, substantially prevent or reduce airflow from a downstream to upstream direction along the passageway d170. In FIG. 19, two valves d166 are located immediately upstream and immediately downstream, respectively, of the nicotine-impregnated substrate d160. In some embodiments, only one valve d166 may be provided. In further embodiments, a valve d166 may be located upstream of the heater d164. In still further embodiments, the valves may be omitted.

Development E: An Aerosol Delivery Apparatus Comprising One-Way Valves both Upstream and Downstream of the Aerosol Generating Part

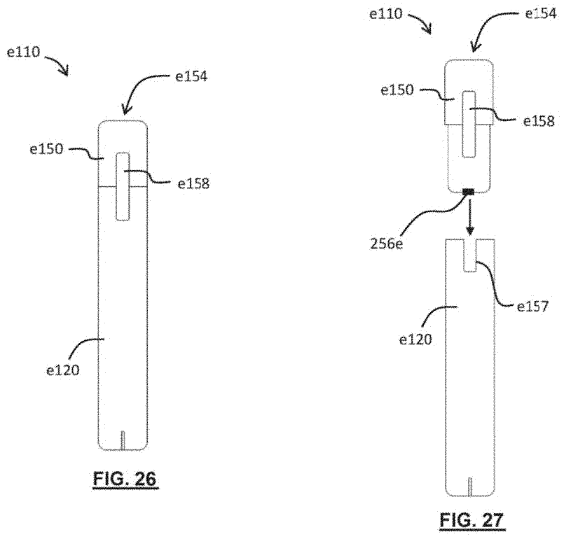

FIGS. 26 and 27 illustrate a smoking substitute system in the form of an e-cigarette system e110. The system e110 comprises a main body e120 of the system e110, and a smoking substitute apparatus in the form of an e-cigarette consumable (or “pod”) e150. In the illustrated embodiment the consumable e150 (sometimes referred to herein as a smoking substitute apparatus) is removable from the main body e120, so as to be a replaceable component of the system e110. The e-cigarette system e110 is a closed system in the sense that it is not intended that the consumable should be refillable with e-liquid by a user.

As is apparent from FIGS. 26 and 27, the consumable e150 is configured to engage the main body e120. FIG. 26 shows the main body e120 and the consumable e150 in an engaged state, whilst FIG. 27 shows the main body e120 and the consumable e150 in a disengaged state. When engaged, a portion of the consumable e150 is received in a cavity of corresponding shape in the main body e120 and is retained in the engaged position by way of a snap-engagement mechanism. In other embodiments, the main body e120 and consumable e150 may be engaged by screwing one into (or onto) the other, or through a bayonet fitting, or by way of an interference fit. In the illustrated embodiment, the consumable e150 is a “single-use” consumable e150. The term “single-use” does not necessarily mean the consumable is designed to be disposed of after a single smoking session. Rather, it defines that the consumable e150 is not designed to be refilled, and is instead disposed of and replaced after usage.

The power source of the main body e120 may be in the form of a battery (e.g., a rechargeable battery such as a lithium ion battery). The main body e120 may comprise a connector in the form of, e.g., a USB port for recharging this battery. The main body e120 may also comprise a controller that controls the supply of power from the power source to the main body electrical contacts (and thus to the filament e264, heater e164 or heating element e364). That is, the controller may be configured to control a voltage applied across the main body electrical contacts, and thus the voltage applied across the heater e164, filament e264 or heating element e364. In this way, the heater e164, filament e264 or heating element e364 may only be heated under certain conditions (e.g., during a puff and/or only when the system is in an active state). In this respect, the main body e120 may include a puff sensor (not shown) that is configured to detect a puff (i.e., inhalation). The puff sensor may be operatively connected to the controller so as to be able to provide a signal, to the controller, which is indicative of a puff state (i.e., puffing or not puffing). The puff sensor may, for example, be in the form of a pressure sensor or an acoustic sensor.

Although not shown, the main body e120 and consumable e150 may comprise a further interface which may, for example, be in the form of an RFID reader, a barcode or QR code reader. This interface may be able to identify a characteristic (e.g., a type) of a consumable e150 engaged with the main body e120. In this respect, the consumable e150 may include any one or more of an RFID chip, a barcode or QR code, or memory within which is an identifier and which can be interrogated via the interface.

The system e110 is configured to vaporise an aerosol precursor, which in the illustrated first embodiment is a nicotine-based liquid impregnated into a substrate e160. The nicotine-impregnated substrate e160 may be referred to as an aerosol generator. When air is drawn through or over the nicotine-impregnated substrate e160, the nicotine is vaporised and entrained in the airflow to thereby be delivered to a user. The vapour or aerosol produced by the aerosol generator is less visible than that produced by a conventional e-liquid from an e-cigarette when exhaled by a user. Preferably, the vapour or aerosol generated by the aerosol generator is invisible or substantially invisible when exhaled by a user.

A substrate e160 may be impregnated with nicotine by immersion in a solution of nicotine in a volatile carrier solvent (e.g., ethanol) such that the substrate e160 is evenly soaked. The substrate e160 can then be removed and left to dry or baked in an oven, meaning that the carrier is evaporated and the nicotine is left evenly spread throughout the substrate.

The nicotine-impregnated substrate e160 may be provided within a consumable e150a. In such an embodiment, when the supply of nicotine in the nicotine-impregnated substrate e160 is depleted, the consumable e150a may be replaced. In other embodiments, the nicotine-impregnated substrate e160 itself may be a consumable component of the system e110. For example, the nicotine-impregnated substrate may be locatable within and removable from the system e110.

Further details are set out above relating to the air-permeable substrate, where used, and its impregnation with an active ingredient under the heading “air-permeable substrate”, those details applying to some embodiments of Development E described here.

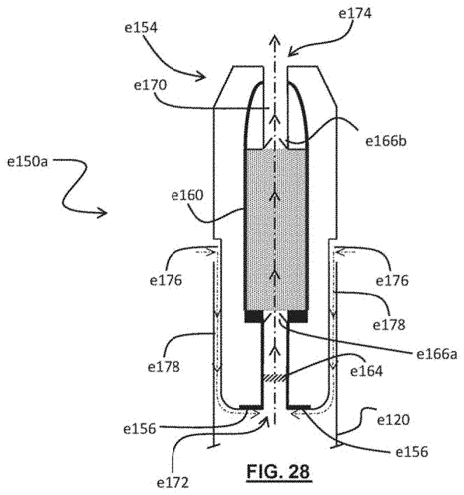

FIG. 28 shows a schematic longitudinal cross-sectional view of the aerosol generator which forms part of the substitute smoking system shown in FIGS. 26 and 27. In FIG. 28, the nicotine-impregnated substrate e160 is arranged within a passage e170. The passage e170 extends between an aerosol generator inlet e172 and an aerosol generator outlet e174 at opposing ends of the consumable e150. In this respect, the passage e170 comprises an upstream end at the end of the consumable e150 that engages with the main body e120, and a downstream end at an opposing end of the consumable e150 that comprises a mouthpiece e154 of the system e110.

When the consumable e150a is received in the cavity of the main body e120 as shown in FIG. 28, a plurality of device air inlets e176 are formed at the boundary between the casing of the consumable and the casing of the main body. The device air inlets e176 are in fluid communication with the aerosol generator inlet e172 through an inlet flow channel e178 formed in the cavity of the main body which is of corresponding shape to receive a part of the consumable e150a. Air from outside of the system e110 can therefore be drawn into the passage e170 through the device air inlets e176 and the inlet flow channels e178.

When the consumable e150a is engaged with the main body e120, a user can inhale (i.e., take a puff) via the mouthpiece e154 so as to draw air through the passage e170, and so as to form an airflow (indicated by the dashed arrows in FIG. 28) in a direction from the aerosol generator inlet e172 to the aerosol generator outlet e174. Air is thereby drawn through and/or around the nicotine-impregnated substrate e160, such that nicotine from the nicotine-impregnated substrate e160 can be entrained in the airflow. The nicotine-impregnated substrate e160 is arranged to extend across a cross-section of at least a portion of the passage e170, such that substantially all of the air drawn through the passage e170 passes through at least a part of the reservoir e160. The resistance to drawing air through the consumable e150a may be configured by altering the air permeability of the nicotine-impregnated substrate, or by the provision of one or more bypass passages separate from the passage e170 (not illustrated).

In this embodiment, a heater e164 is arranged upstream of the nicotine-impregnated substrate e160 along the passageway e170. The heater e164 is operable to heat air passing through the passageway e170 to enable or enhance nicotine entrainment. In this embodiment, the heater e164 comprises an electrically heatable mesh located in the airflow stream. The mesh may be formed of a material that is heatable by resistive heating using an electrical current. In other embodiments, the heater e164 may comprise a plurality of meshes. The heater e164 may be located in a passage in the base unit e120 (not illustrated), with the base unit passageway being in fluid communication with the passageway e170 of the consumable e150a. In further embodiments, the heater e164 may be located externally of the passageway e170 but placed in thermal communication with the airflow via thermally conductive elements which extend into or across the passageway e170 (not illustrated). In still further embodiments, the heater may be omitted.

When the consumable e150a is engaged with the main body e120, electrical contacts e156 make contact with corresponding electrical contacts (not shown) of the main body e120. The main body electrical contacts are electrically connectable to a power source (not shown) of the main body e120, such that (in the engaged position) the heater e164 is electrically connectable to the power source. In this way, power can be supplied by the main body e120 to the heater e164 in order to heat the heater e164.

The system e110 may alternatively be configured to vaporise an aerosol precursor, which in the illustrated second embodiment is in the form of a nicotine-based e-liquid e260. Components of the system e110 which are common with the first embodiment are referred to by the same reference numeral, and will not be further explained. The e-liquid e260 comprises nicotine and a base liquid including propylene glycol and/or vegetable glycerine. In the present embodiment, the e-liquid e260 is flavoured by a flavourant. In other embodiments, the e-liquid e260 may be flavourless and thus may not include any added flavourant.

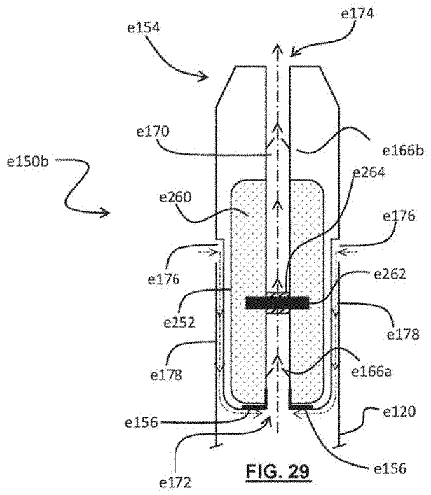

The aerosol precursor is vaporised by the aerosol generator of the system e110. FIG. 29 shows a schematic longitudinal cross-sectional view of the aerosol generator which can form part of the substitute smoking system shown in FIGS. 26 and 27. In FIG. 29, the e-liquid e260 is stored within a reservoir in the form of a tank e252 that forms part of the system e110 or a consumable e150b.

The tank may include a vent (not shown) to allow ingress of air to replace e-liquid that has been used from the tank. The consumable e150b preferably includes a window e158 (see FIGS. 26 and 27), so that the amount of e-liquid in the tank e252 can be visually assessed. The main body e120 includes a slot e157 so that the window e158 of the consumable e150b can be seen whilst the rest of the tank e252 is obscured from view when the consumable e150b is received in the cavity of the main body e120. The consumable e150b may be referred to as a “clearomizer” when it includes a window e158, or a “cartomizer” when it does not.

In other embodiments, the tank may be refillable with e-liquid or the e-liquid may be stored in a non-consumable component of the system. For example, in such other embodiments, the e-liquid may be stored in a tank located in the main body or stored in another component that is itself not single-use (e.g., a refillable cartomizer).

In FIG. 29, the tank e252 annularly surrounds, and thus defines a portion of, a passage e170 that extends between an aerosol generator inlet e172 and an outlet e174 at opposing ends of the consumable e150b. In this respect, the passage e170 comprises an upstream end at the end of the consumable e150b that engages with the main body e120, and a downstream end at an opposing end of the consumable e150b that comprises a mouthpiece e154 of the system e110.

When the consumable e150b is received in the cavity of the main body e120 as shown in FIG. 29, a plurality of device air inlets e176 are formed at the boundary between the casing of the consumable and the casing of the main body. The device air inlets e176 are in fluid communication with the aerosol generator inlet e172 through an inlet flow channel e178 formed in the cavity of the main body which is of corresponding shape to receive a part of the consumable e150b. Air from outside of the system e110 can therefore be drawn into the passage e170 through the device air inlets e176 and the inlet flow channels e178.

When the consumable e150b is engaged with the main body e120, a user can inhale (i.e., take a puff) via the mouthpiece e154 so as to draw air through the passage e170, and so as to form an airflow (indicated by the dashed arrows in FIG. 29) in a direction from the aerosol generator inlet e172 to the outlet e174. Although not illustrated, the passage e170 may be partially defined by a tube (e.g., a metal tube) extending through the consumable e150b. In FIG. 29, for simplicity, the passage e170 is shown with a substantially circular cross-sectional profile with a constant diameter along its length. In other embodiments, the passage may have other cross-sectional profiles, such as oval shaped, racetrack shaped or polygonal shaped profiles. Further, in other embodiments, the cross sectional profile and the diameter (or hydraulic diameter) of the passage may vary along its longitudinal axis.

The aerosol generator of the smoking substitute system e110 is configured to vaporise the e-liquid e260 for inhalation by a user. To provide this operability, the aerosol generator comprises a heater having a porous wick e262 and a resistive heating element in the form of a heating filament e264 that is helically wound (in the form of a coil) around a portion of the porous wick e262. The porous wick e262 extends across the passage e170 (i.e., transverse to a longitudinal axis of the passage e170 and thus also transverse to the air flow along the passage e170 during use) and opposing ends of the wick e262 extend into the tank e252 (so as to be immersed in the e-liquid e260). In this way, e-liquid e260 contained in the tank e252 is conveyed from the opposing ends of the porous wick e262 to a central portion of the porous wick e262 so as to be exposed to the airflow in the passage e170.

The helical filament e264 is wound about the exposed central portion of the porous wick e262 and is electrically connected to an electrical interface in the form of electrical contacts e156 mounted at the end of the consumable that is proximate the main body e120 (when the consumable and the main body are engaged). When the consumable e150b is engaged with the main body e120, electrical contacts e156 make contact with corresponding electrical contacts (not shown) of the main body e120. The main body electrical contacts are electrically connectable to a power source (not shown) of the main body e120, such that (in the engaged position) the filament e264 is electrically connectable to the power source. In this way, power can be supplied by the main body e120 to the filament e264 in order to heat the filament e264. This heats the porous wick e262 which causes e-liquid e260 conveyed by the porous wick e262 to vaporise and thus to be released from the porous wick e262. The vaporised e-liquid becomes entrained in the airflow and, as it cools in the airflow (between the heated wick and the outlet e174 of the passage e170), condenses to form an aerosol. This aerosol is then inhaled, via the mouthpiece e154, by a user of the system e110. As e-liquid is lost from the heated portion of the wick, further e-liquid is drawn along the wick from the tank to replace the e-liquid lost from the heated portion of the wick.

The filament e264 and the exposed central portion of the porous wick e262 are positioned across the passage e170. More specifically, the part of passage that contains the filament e264 and the exposed portion of the porous wick e262 forms a vaporisation chamber. In the illustrated example, the vaporisation chamber has the same cross-sectional diameter as the passage e170. However, in other embodiments the vaporisation chamber may have a different cross sectional profile as the passage e170. For example, the vaporisation chamber may have a larger cross sectional diameter than at least some of the downstream part of the passage e170 so as to enable a longer residence time for the air inside the vaporisation chamber.

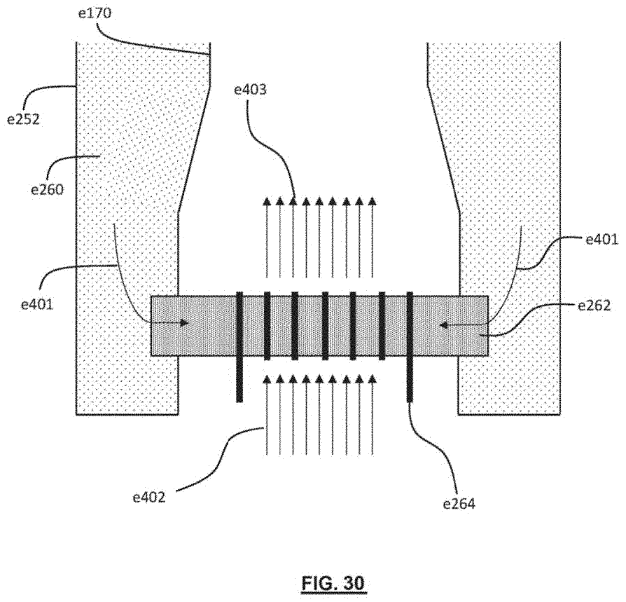

FIG. 30 illustrates in more detail the vaporisation chamber and therefore the region of the consumable e150 around the wick e262 and filament e264. The helical filament e264 is wound around a central portion of the porous wick e262. The porous wick extends across passage e170. E-liquid e260 contained within the tank e252 is conveyed as illustrated schematically by arrows e401, i.e., from the tank and towards the central portion of the porous wick e262.

When the user inhales, air is drawn from through the inlets e176 shown in FIG. 29, along inlet flow channel e178 to vaporisation chamber inlet e172 and into the vaporisation chamber containing porous wick e262. The porous wick e262 extends substantially transverse to the airflow direction. The airflow passes around the porous wick, at least a portion of the airflow substantially following the surface of the porous wick e262. In examples where the porous wick has a cylindrical cross-sectional profile, the airflow may follow a curved path around an outer periphery of the porous wick e262.

At substantially the same time as the airflow passes around the porous wick e262, the filament e264 is heated so as to vaporise the e-liquid which has been wicked into the porous wick. The airflow passing around the porous wick e262 picks up this vaporised e-liquid, and the vapour-containing airflow is drawn in direction e403 further down passage e170.

The system e110 may alternatively generate vapour via heating of tobacco material in the form of an aerosol forming substrate e360, as shown in the illustrated third embodiment of FIG. 31. Components of the system e110 which are common with the first and second embodiments are referred to by the same reference numeral, and will not be further explained. The aerosol forming substrate e360 comprises tobacco material that may, for example, include any suitable parts of the tobacco plant (e.g., leaves, stems, roots, bark, seeds and flowers). In order to generate an aerosol, the aerosol forming substrate e360 comprises at least one volatile compound that is intended to be vaporised/aerosolised and that may provide the user with a recreational and/or medicinal effect when inhaled. The aerosol-forming substrate e360 may further comprise one or more additives. For example, such additives may be in the form of humectants (e.g., propylene glycol and/or vegetable glycerine), flavourants, fillers, aqueous/non-aqueous solvents and/or binders.

The system e110 is configured to heat the aerosol-forming substrate e360 so as to form an aerosol for inhalation by a user. To provide this operability, the aerosol generator comprises a heating element e364 that projects into the aerosol-forming substrate e360. This heating element e364 is electrically connected to a power supply (not shown) of the system e110 and, when activated, heats the aerosol-forming substrate e360 such that vapour is released from the aerosol-forming substrate e360. When a user inhales via the mouth end e154, air is drawn through the heated aerosol-forming substrate e360 and the vapour becomes entrained in the resultant airflow. As the vapour flows from the aerosol-forming substrate e360 to the downstream end e172 of the passage e170, it condenses into an aerosol and the aerosol is inhaled by the user.

In each of the foregoing embodiments, the passageway e170 of the smoking substitute apparatus e150a, e150b, e150c comprises two or more one-way valves e166. The valves serve to control airflow through the passageway e170. More specifically, the valves are provided to prevent, substantially prevent or reduce airflow from a downstream to upstream direction along the passageway e170. A one-way valve may also be referred to as a check valve. As illustrated in FIGS. 28, 29, 31, a first valve e166a is located upstream of the aerosol generator, and a second valve e166a is located downstream of the aerosol generator.

The valves e166a, e166b may be located immediately upstream and immediately downstream of the aerosol generator. Providing valves e166a, e166b immediately upstream and immediately downstream of the nicotine-impregnated substrate e160 is advantageous for preventing nicotine or nicotine infused liquid from escaping from the nicotine-impregnated substrate e160 when the smoking substitute apparatus e150 is not in use.

In embodiments where a heater e164 is provided to heat air in the air passage e170, the upstream valve e166a may be located upstream of the heater e164. Providing a valve e166a upstream of a heater e164 allows control over the airflow, while not requiring the valve to be suitable to operate with heated air. A valve e166a provided upstream of a heater e164 may also improve the efficiency of utilisation for the heater, since heated air is prevented from flowing in an upstream direction from the heater e164.

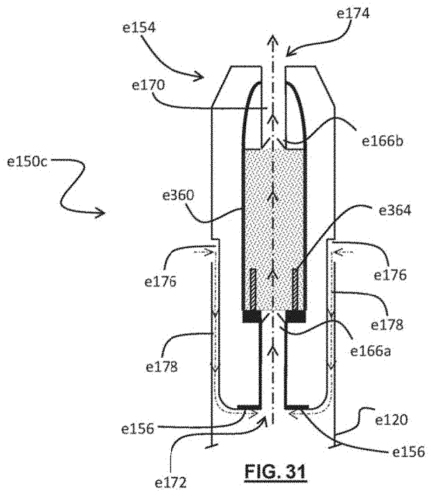

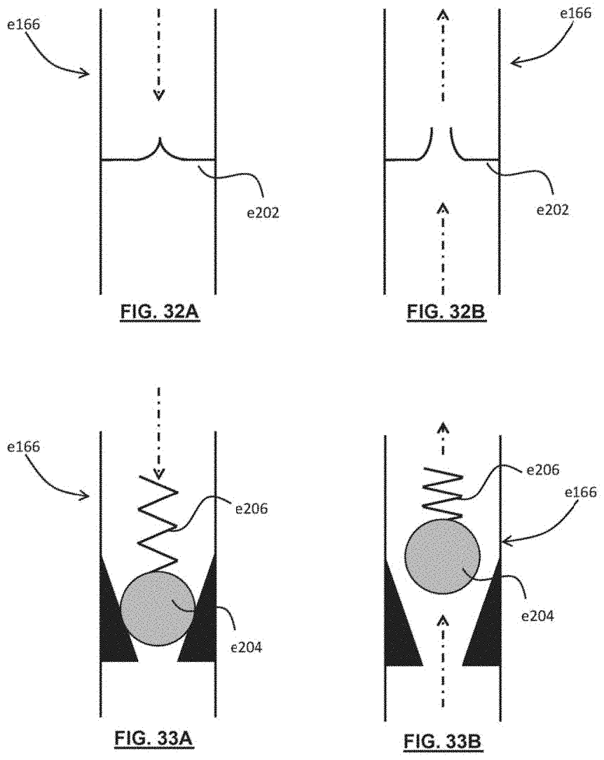

At least one of the one-way valves e166 may be a duckbill valve, as illustrated exemplarily in FIGS. 32A, 32B. A duckbill valve comprises an elastomeric material diaphragm e202 in which a portion is shaped like the bill or beak of a duck, comprising an opening or aperture therethrough. Fluid flow in the “allowed” direction opens the duckbill shaped portion (FIG. 32B), while fluid flow in a reverse direction (or absence of any fluid flow) cases the duckbill shaped portion to close (FIG. 32A). A pressure differential across the valve e166 from the upstream to downstream side may be required to open the valve and allow fluid to flow.

At least one of the one-way valves e166 may be a ball one-way valve, as illustrated exemplarily in FIGS. 33A and 33B. A ball one-way valve comprises a ball e204 located in a tapered passage so as to prevent fluid flow in a reverse direction (FIG. 33A). Fluid flow in an allowed direction moves the ball e204, allowing flow around the ball e204 (FIG. 33B). A ball one-way valve may further comprise a spring e206 to position the ball e204 within the valve. In some embodiments, the spring e206 may be omitted. As with the duckbill valve, a one-way ball valve may require a pressure differential across the valve e166 in an upstream to downstream direction to open the valve e166 and allow flow through the valve e166, particularly in valves where a spring e206 is present.

The features disclosed in the foregoing description, or in the following claims, or clauses, or in the accompanying drawings, expressed in their specific forms or in terms of a means for performing the disclosed function, or a method or process for obtaining the disclosed results, as appropriate, may, separately, or in any combination of such features, be utilised for realising the disclosure in diverse forms thereof.

While the disclosure has been described in conjunction with the exemplary embodiments described above, many equivalent modifications and variations will be apparent to those skilled in the art when given this disclosure. Accordingly, the exemplary embodiments of the disclosure set forth above are considered to be illustrative and not limiting. Various changes to the described embodiments may be made without departing from the spirit and scope of the disclosure.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the words “have”, “comprise”, and “include”, and variations such as “having”, “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means, for example, −/−10%.

The words “preferred” and “preferably” are used herein refer to embodiments of the disclosure that may provide certain benefits under some circumstances. It is to be appreciated, however, that other embodiments may also be preferred under the same or different circumstances. The recitation of one or more preferred embodiments therefore does not mean or imply that other embodiments are not useful, and is not intended to exclude other embodiments from the scope of the disclosure, or from the scope of the claims.

### Illustrative Embodiments

In the following numbered “clauses” are set out statements of broad combinations of novel and inventive features of the present disclosure herein disclosed.

A1. An aerosol delivery apparatus (150) comprising:
an air passage (170);
a reservoir (160) formed from an air-permeable substrate and arranged to allow air to be drawn through the reservoir (160), the reservoir (160) being loaded with a source of an active ingredient; and
one or more one-way valves (166) arranged along the air passage (170) and configured to allow air to flow along the air passage (170) in an upstream to downstream direction.

A2. An aerosol delivery apparatus (150) according to clause A1, wherein at least one of the one or more one-way valves (166) is:
arranged upstream of the reservoir (160) along the air passage (170); or
arranged downstream of the reservoir (160) along the air passage (170).

A3. An aerosol delivery apparatus (150) according to either of clauses A1 or A2, wherein:
the apparatus (160) comprises a first one-way valve (166a) and a second one-way valve (166b) arranged within the air passage (170), and wherein
the first one-way valve (166a) is arranged upstream of the reservoir along the air passage (170); and
the second one-way valve (166b) is arranged downstream of the reservoir along the air passage (170).

A4. An aerosol delivery apparatus (150) according to any preceding clause A1 to A3, further comprising: a heater (164) arranged in the air passage (170) and upstream of the reservoir, the heater (164) being operable to heat air passing through the air passage (170).

A5. An aerosol delivery apparatus (150) according to clause A4, wherein: the heater (164) comprises an electrically heatable mesh.

A6. An aerosol delivery apparatus (150) according to any one of clauses A4 to A5, wherein: the heater (164) is heatable by resistive heating using an electrical current.

A7. An aerosol delivery apparatus (160) according to any of clauses A4 to A6, wherein: a one-way valve (166) of the one-way valves (166) is arranged upstream of the heater (164) along the air passage (170).

A8. An aerosol delivery apparatus (150) according to any preceding clause A1 to A7, wherein: a one-way valve (166) of the one-way valves (166) is a duckbill valve.

A9. An aerosol delivery apparatus (150) according to any preceding clause A1 to A8, wherein: a one-way valve (166) of the one-way valves (166) is a ball one-way valve.

A10. An aerosol delivery apparatus (150) according to clause A9, wherein: the ball one-way valve (166) further comprises a spring (206) to locate the ball within the valve (166).

A11 . An aerosol delivery apparatus (150) according to any preceding clause A1 to A10, wherein: the combined resistance to draw presented by the one-way valve (166), reservoir (160) and air passage (170) is substantially equal to that of a conventional cigarette.

A12. An aerosol delivery apparatus (150) according to any preceding clause A1 to A11, wherein: the reservoir (160) is a consumable component of the aerosol delivery apparatus (150).

A13. An aerosol delivery apparatus (150) according to any one of clauses A1 to A12, wherein the aerosol delivery apparatus (150) is comprised by or within a cartridge configured for engagement with a base unit (120), the cartridge and base unit together forming an aerosol delivery system (110).

A14. An aerosol delivery system (110) comprising:
a base unit (120), and
an aerosol delivery apparatus (150) according to clause A13, wherein the aerosol delivery apparatus (150) is removably engageable with the base unit (120).

A15. A method of using an aerosol delivery apparatus (150) according to any one of clauses A1 to A13 to generate an aerosol.

B1. An aerosol delivery system (110) comprising:
an air inlet (172);
an outlet (174);
an air passage (170) extending from the air inlet (172) to the outlet (174);
a part of the air passage being defined by a heat source air passage (170a) extending through a heat source (200) operable to heat air in the heat source air passage (170a) through an exothermic reaction; and
a reservoir (160) formed from an air-permeable substrate and arranged to allow air in the air passage drawn from the outlet (174) of the air passage (170) to be drawn through the reservoir (160) and on to the outlet (174), the reservoir (160) being loaded with a source of an active ingredient; wherein
the heat source air passage (170a) has a heat source air inlet and a heat source air outlet, spaced apart on the heat source (200) by a separation distance, wherein, in use, the heat source air passage (170a) conveys air along a flow path within the heat source (200) from the heat source air inlet to the heat source air outlet, and wherein the length of the flow path is greater than the separation distance.

B2. An aerosol delivery system (110) according to clause B1, wherein: the heat source air passage (170a) conveys air along a tortuous flow path.

B3. An aerosol delivery system (110) according to either one of clause B1 or clause B2, wherein: the smoking substitute system (110) comprises a plurality of separate heat source air passages (170a).

B4. An aerosol delivery apparatus (150) according to clause B3, wherein: at least two of the heat source air passages (170a) extend from a common heat source air inlet and/or to a common heat source air outlet.

B5. An aerosol delivery system (110) according to any preceding clause B1 to B4, wherein: the exothermic reaction of the heat source (200) is a crystallisation of a super-saturated solution.

B6. An aerosol delivery system (110) according clause B5, wherein: the exothermic reaction of the heat source (200) is initiable by operation of a multistable trigger element.

B7. An aerosol delivery system (110) according to any one of clauses B1 to B4, wherein: the exothermic reaction of the heat source (200) is an oxidation reaction.

B8. An aerosol delivery system (110) according to clause B7, wherein: the exothermic reaction is initiable by removal of an oxygen impermeable seal from a reaction chamber of the heat source (200).

B9. An aerosol delivery system (110) according to any one of clauses B1 to B4, wherein: the exothermic reaction of the heat source (200) is a reaction between two or more substances stored inside the heat source (200) and initiable by contact between the two or more substances.

B10. An aerosol delivery system according to any preceding clause B1 to B9, wherein the aerosol delivery system is a smoking substitute system and the active ingredient is nicotine.

B11. An aerosol delivery system (110) according to any preceding clause B1 to B10, wherein: the heat source (200) is a replaceable component of the aerosol delivery system (110).

B12. A kit of parts for an aerosol delivery system (110) according to any preceding clause B1 to B11, comprising:
a base unit (120);
an aerosol delivery apparatus (150) removably engageable with the base unit (120); and
the heat source (200); wherein:
the aerosol delivery apparatus (150) is a cartridge configured for engagement with the base unit (120), the cartridge comprising the reservoir (160).

B13. A kit of parts for an aerosol delivery system (110) according to clause B12, wherein, in use, the heat source (200) is comprised within the base unit (120).

B14. A kit of parts for an aerosol delivery system (110) according to clause B12, wherein, in use, the heat source (200) is comprised within the aerosol delivery apparatus (150).

B15. A method of using an aerosol delivery system according to any of clauses B1 to B11, comprising the steps of:
initiating the exothermic reaction; and
drawing air through the passage to generate heated air in the heat source air passage and through the reservoir to generate a vapour.

C1. An aerosol delivery apparatus (150b) comprising:
a first reservoir region (160a) formed from an air-permeable substrate and arranged to allow air to be drawn through the first reservoir region (160a), the first reservoir region (160a) being loaded with a source of nicotine and being located in a first spatial location in the aerosol delivery apparatus (150b), and
a second reservoir region (160b) formed from a substrate and arranged to allow air to be drawn in contact with the second reservoir region (160b), the second reservoir region (160b) being loaded with a source of flavourant and being located in a second spatial location in the aerosol delivery apparatus (150b),
wherein the first and second spatial locations are not coterminous.

C2. An aerosol delivery apparatus (150b) according to clause C1, wherein: the first reservoir region (160a) and the second reservoir region (160b) are comprised within a monolithic substrate.

C3. An aerosol delivery apparatus (150b) according to clause C1, wherein: the first reservoir region (160a) is comprised within a first substrate, and the second reservoir region (160b) is comprised within a second substrate.

C4. An aerosol delivery apparatus (150b) according to clause C3, wherein: the first reservoir region (160a) and the second reservoir region (160b) are arranged within the aerosol delivery apparatus (150b) such that a gap is defined between the substrate of the first reservoir region (160a) and the substrate of the second reservoir region (160b).

C5. An aerosol delivery apparatus (150b) according to any preceding clause C1 to C4, wherein: the first spatial location and the second spatial location are separated along a direction parallel to the direction of air being drawn through the first (160a) and second reservoir region (160b).

C6. An aerosol delivery apparatus (150b) according to any preceding clause C1 to C5, wherein: the first spatial location and the second spatial location are separated along a direction perpendicular to the direction of air being drawn through first (160a) and second reservoir region (160b).

C7. An aerosol delivery apparatus (150b) according to any preceding clause C1 to C6, further comprising:
an air inlet (176); and
a passage (170) leading from the air inlet (176) to a first outlet (174); wherein
the first reservoir region (160a) and the second reservoir region (160b) are arranged within the passage (170).

C8. An aerosol delivery apparatus according to clause C7, further comprising: a heater (164) arranged in the passage (170) and configured to heat air being drawn through the first (160a) and second (160b) reservoir regions.

C9. An aerosol delivery apparatus according to either of clauses C7 or C8, further comprising: a one-way valve (166) arranged along the air passage (170) and configured to allow air to flow along the air passage (170) in an upstream to downstream direction.

C10. An aerosol delivery apparatus (150b) according to any of clauses C7 to C9, wherein: the first (160a) and second (160b) reservoir regions are consumable components of the aerosol delivery apparatus.

C11. An aerosol delivery apparatus (150b) according to clause C10, further comprising: an encapsulating material layer enclosing the first (160a) and second (160b) reservoir regions.

C12. A consumable kit of parts adapted for use in an aerosol delivery apparatus (150b) according to any of clauses C10 or C11, comprising:
a first reservoir region (160a) formed from an air-permeable substrate, the first reservoir region (160a) being loaded with a source of nicotine, and
a second reservoir region (160b) formed from a substrate, the second reservoir region (160b) being loaded with a source of flavourant,
wherein the first (160a) and second (160b) reservoir regions are not coterminous.

C13. An aerosol delivery apparatus (150b) according to any one of clause C1 to C11, wherein the smoking substitute apparatus (150b) is comprised by or within a cartridge configured for engagement with a base unit (120), the cartridge and base unit together forming an aerosol delivery system (110).

C14. An aerosol delivery system (110) comprising:
a base unit (120), and
an aerosol delivery apparatus (150b) according to clause C13, wherein the aerosol delivery apparatus (150b) is removably engageable with the base unit (120).

C15. A method of using an aerosol delivery apparatus (150b) according to any one of clauses C1 to C11 to generate an aerosol.

D1. A smoking substitute system (110) comprising:
an air passage (170);
a reservoir (160) formed from an air-permeable substrate and arranged in the air passage (170) such that substantially all of the air drawn through the passage (170) is drawn through the reservoir (160), the air-permeable substrate being loaded with substantially pure nicotine; and
a heater (164) arranged in the air passage (170) and upstream of the reservoir (160), the heater (164) being operable to heat air passing through the air passage (170).

D2. A smoking substitute system (110) according to clause D1, wherein: the heater (164) for heating the air in the air passage (170) comprises an electrically heatable mesh.

D3. A smoking substitute system (110) according to clause D2, wherein: the electrically heatable mesh is formed as a sheet and the sheet is configured in a helical arrangement extending along the passage (170).

D4. A smoking substitute system (110) according to any one of clauses D2 or D3, wherein: the heater (164) for heating the air in the air passage (170) comprises a plurality of meshes having a common electrical connection.

D5. A smoking substitute system (110) according to any one of clauses D2 to D4, wherein: the heater (164) for heating the air in the air passage (170) comprises a plurality of meshes connected in series.

D6. A smoking substitute system (110) according to any one of clauses D2 to D5, wherein: the heater (164) for heating the air in the air passage (170) comprises a plurality of meshes connected in parallel.

D7. A smoking substitute system (110) according to any one of clauses D2 to D6, wherein the heater (164) for heating the air in the air passage (170) comprises a plurality of meshes which are independently operable.

D8. A smoking substitute system (110) according to any preceding clause D1 to D7, wherein: the heater (164) for heating the air in the air passage (170) comprises an electrically heatable heating coil.

D9. A smoking substitute system (110) according to any preceding clause D1 to D8, wherein: the heater (164) for heating the air in the air passage (170) comprises an electrically heatable heating plate.

D10. A smoking substitute system (110) according to clause D9, wherein: the heating plate surrounds and defines a part of the air passage (170).

D11. A smoking substitute system (110) according to any preceding clause D1 to D10, wherein: the heater (164) for heating the air in the air passage (170) is heatable by resistive heating using an electrical current.

D12. A smoking substitute system (110) according to any preceding clause D1 to D11, wherein: one or more one-way valves (166) arranged along the air passage (170) and configured to allow air to flow along the air passage (170) in an upstream to downstream direction.

D13. A kit of parts for a smoking substitute system (110) according to any one of clauses D1 to D12, comprising:
a base unit (120), and
a smoking substitute apparatus (150) removably engageable with the base unit (120);
   wherein:
the base unit (120) comprises a base unit air passage, said base unit air passage comprising the heater (164); and wherein:
the smoking substitute apparatus (150) is a cartridge configured for engagement with the base unit (120), the cartridge comprising a cartridge air passage in fluid communication with the base unit air passage to collectively form the air passage (170) of the smoking substitute system (110), said cartridge air passage comprising the reservoir (160).

D14. A kit of parts for a smoking substitute system (110) according to any one of clauses D1 to D12, comprising:
a base unit (120), and
a smoking substitute apparatus (150) removably engageable with the base unit (120);
   wherein:
the smoking substitute apparatus (150) is a cartridge configured for engagement with the base unit (120), the cartridge comprising at least the portion of the air passage (170) that comprises the heater (164) and the reservoir (160) of the smoking substitute system (110).

D15. A method of using a smoking substitute system (110) according to any one of clauses D1 to D12 to generate an aerosol.

E1. An aerosol delivery apparatus (150a, 150b, 150c) comprising:

an air passage (170);
an aerosol generator arranged in the air passage (170);
a first one-way valve (166a) arranged upstream of the aerosol generator along the air passage (170), and configured to allow air to flow along the air passage (170) in an upstream to downstream direction; and
a second one-way valve (166b) arranged downstream of the aerosol generator along the air passage (170), and configured to allow air to flow along the air passage (170) in an upstream to downstream direction.

E2. An aerosol delivery apparatus (150a, 150b, 150c) according to clause E1, wherein: a one-way valve (166) of the one-way valves (166) is a duckbill valve.

E3. An aerosol delivery apparatus (150a, 150b, 150c) according to either of clauses E1 or E2, wherein: a one-way valve (166) of the one-way valves (166) is a ball one-way valve.

E4. An aerosol delivery apparatus (150a, 150b, 150c) according to clause E3, wherein: the ball one-way valve (166) further comprises a spring (206) to locate the ball within the valve (166).

E5. An aerosol delivery apparatus (150a) according to any preceding clause E1 to E4, wherein: the aerosol generator comprises a reservoir (160) formed from an air-permeable substrate and arranged in the air passage (170) to allow air to be drawn through the reservoir (160), the reservoir (160) being loaded with a source of an active ingredient.

E6. An aerosol delivery apparatus (150a) according to clause E5, further comprising: a heater (164) arranged in the air passage (170) and upstream of the reservoir (160), the heater (164) being operable to heat air passing through the air passage (170).

E7. An aerosol delivery apparatus (150a) according to clause E6, wherein: the heater (164) for heating the air in the air passage (170) comprises an electrically heatable mesh.

E8. An aerosol delivery apparatus (150b) according to any of clauses E1 to E4, wherein: the aerosol generator comprises a porous wick (262) which, in use, wicks aerosol precursor (260) from a reservoir (252) to the first passage (170) for entrainment in air flowing downstream of the aerosol generator.

E9. An aerosol delivery apparatus (150b) according to clause E8, further comprising: a heater operable to generate the aerosol from the aerosol precursor (260), the heater being a heating filament (264) that is wound around a portion of the porous wick (262).

E10. An aerosol delivery apparatus (150b) according to either of clauses E8 or E9, wherein:
the passage (170) comprises a vaporisation chamber in which the aerosol generator is arranged, and wherein
the vaporisation chamber has a larger cross sectional diameter than a downstream part of the passage (170).

E11. An aerosol delivery apparatus (150c) according to any one of clauses E1 to E4, wherein:
the aerosol generator comprises an aerosol-forming substrate (360) comprising tobacco material; and
a heating element (364) operable to heat the aerosol-forming substrate (360) to generate an aerosol for entrainment in air flowing downstream of the aerosol generator.

E12. An aerosol delivery apparatus (150c) according to clause E11, wherein: the heating element (364) comprises an electrically heatable rod.

E13. An aerosol delivery apparatus (150a, 150b, 150c) according to any one of clause E1 to clause E12, wherein the aerosol delivery apparatus (150a, 150b, 150c) is comprised by or within a cartridge configured for engagement with a base unit (120), the cartridge and base unit together forming an aerosol delivery system (110).

E14. An aerosol delivery system (110) comprising:
a base unit (120), and
an aerosol delivery apparatus (150a, 150b, 150c) according to clause E13, wherein the aerosol delivery apparatus (150a, 150b, 150c) is removably engageable with the base unit (120).

E15. A method of using an aerosol delivery apparatus (150a, 150b, 150c) according to any one of clauses E1 to E13 to generate an aerosol.

What is claimed is:

1. An aerosol delivery apparatus comprising:
an air passage;
a reservoir formed from an air-permeable substrate and arranged to allow air to be drawn through the reservoir, the reservoir being loaded with a source of an active ingredient; and
one or more one-way valves arranged along the air passage and configured to allow air to flow along the air passage in an upstream to downstream direction.

2. An aerosol delivery apparatus according to claim 1, wherein at least one of the one or more one-way valves is:
arranged upstream of the reservoir along the air passage; or
arranged downstream of the reservoir along the air passage.

3. An aerosol delivery apparatus according to either of claim 1 or 2, wherein:
the apparatus comprises a first one-way valve and a second one-way valve arranged within the air passage, and wherein
the first one-way valve is arranged upstream of the reservoir along the air passage; and
the second one-way valve is arranged downstream of the reservoir along the air passage.

4. An aerosol delivery apparatus according to any preceding claim, further comprising:
a heater arranged in the air passage and upstream of the reservoir, the heater being operable to heat air passing through the air passage.

5. An aerosol delivery apparatus according to claim 4, wherein: the heater comprises an electrically heatable mesh.

6. An aerosol delivery apparatus according to any one of claims 4 to 5, wherein: the heater is heatable by resistive heating using an electrical current.

7. An aerosol delivery apparatus according to any of claims 4 to 6, wherein: a one-way valve of the one-way valves is arranged upstream of the heater along the air passage.

8. An aerosol delivery apparatus according to any preceding claim, wherein: a one-way valve of the one-way valves is a duckbill valve.

9. An aerosol delivery apparatus according to any preceding claim, wherein: a one-way valve of the one-way valves is a ball one-way valve.

10. An aerosol delivery apparatus according to claim 9, wherein: the ball one-way valve further comprises a spring to locate the ball within the valve.

11. An aerosol delivery apparatus according to any preceding claim, wherein: the combined resistance to draw presented by the one-way valve, reservoir and air passage is substantially equal to that of a conventional cigarette.

12. An aerosol delivery apparatus according to any preceding claim, wherein: the reservoir is a consumable component of the aerosol delivery apparatus.

13. An aerosol delivery apparatus according to any one of claims 1 to 12, wherein the aerosol delivery apparatus is comprised by or within a cartridge configured for engagement with a base unit, the cartridge and base unit together forming an aerosol delivery system.

14. An aerosol delivery system comprising:
a base unit, and
an aerosol delivery apparatus according to claim 13, wherein the aerosol delivery apparatus is removably engageable with the base unit.

15. A method of using an aerosol delivery apparatus according to any one of claims 1 to 13 to generate an aerosol.
